(19) United States
(12) Patent Application Publication
(10) Pub. No.: US 2018/0282783 A1
(43) Pub. Date: Oct. 4, 2018

(54) OLIGONUCLEOTIDE-BASED PROBES FOR DETECTION OF CIRCULATING TUMOR CELL NUCLEASES

(71) Applicant: UNIVERSITY OF IOWA RESEARCH FOUNDATION, Iowa City, IA (US)

(72) Inventors: Paloma H. Giangrande, Iowa City, IA (US); James O. McNamara, Iowa City, IA (US); David D. Dickey, Iowa City, IA (US); Howard Ozer, Chicago, IL (US); Sven Kruspe, Iowa City, IA (US)

(73) Assignee: UNIVERSITY OF IOWA RESEARCH FOUNDATION, Iowa City, IA (US)

(21) Appl. No.: 15/569,002

(22) PCT Filed: Apr. 25, 2016
(86) PCT No.: PCT/US16/29201

§ 371 Date: Oct. 24, 2017

Related U.S. Application Data
(60) Provisional application No. 62/152,750, filed on Apr. 24, 2015.

(51) Int. Cl. C12Q 1/44; C12Q 1/6823; C12Q 1/6886

(52) U.S. Cl. CPC C12Q 1/44; C12Q 2600/112; C12Q 1/6886; C12Q 1/6823

(57) ABSTRACT

The present invention relates to a rapid detection of circulating tumor cell (CTC)-associated nuclease activity with chemically modified nuclease substrate probes and compositions useful in detection assays.

### RELATED APPLICATION

This application claims priority to U.S. Provisional Patent Application No. 62/152,750 filed Apr. 24, 2015, the entirety of which is incorporated herein by reference.

### BACKGROUND OF THE INVENTION

Approximately 12% of US women will develop invasive breast cancer over the course of their lives. There were approximately 300,000 new cases of breast cancer diagnosed in the US in 2014. Metastatic breast cancer (MBC) is breast cancer that has spread beyond the breast and axillary lymph nodes to other organs (most commonly, bones, lungs, liver, or the brain). MBC can be managed, but cannot be cured. It is estimated that 20-30% of breast cancers will become metastatic. MBC is responsible for approximately 90% of deaths from breast cancer and is the second most common cause of death from cancer among US women. The median survival rate after diagnoses is three years.

During the progression of metastasis, cancer cells detach from the solid primary tumor, enter the blood stream, and travel to different tissues of the body. These breakaway cancer cells in the peripheral blood are called circulating tumor cells (CTCs). CTCs constitute seeds for subsequent growth of additional tumors (metastasis) in other areas of the body, a primary cause of death for those with cancer. CTCs are very rare, often present at a concentration of 1 to 10 CTCs per mL of blood in patients with metastases. High levels of CTCs correlate with lower survival (FIG. 1). Testing for CTCs could provide for the early and non-invasive detection of cancer. Further, additional characterization of CTCs could give biological insights into cancer properties for better treatment.

Currently, there is one method (CELLSEARCH® by Johnson and Johnson) that is FDA approved to detect CTCs. It requires separating a blood sample via centrifuge, removal of the plasma, and then enriching the sample with specific antibodies targeting the epithelial cell adhesion molecule. Tumor cells of epithelial origin (including metastatic breast cancer cells) are then magnetically separated. Then, various stains are applied and the tumor cells magnetically moved to one focal depth. The sample is then scanned in a florescence optical system. However, this test is labor-intensive, time-consuming, expensive, and requires a high level of expertise to perform. Additionally, it has high false positives and false negatives.

Accordingly, a rapid, inexpensive, CTC-specific assay is needed.

### SUMMARY OF THE INVENTION

Accordingly, in certain embodiments, the present invention provides a substrate probe for detecting a circulating tumor cell (CTC) nuclease comprising an oligonucleotide of 2-30 nucleotides in length, a fluorophore operably linked to the oligonucleotide, and a quencher operably linked to the oligonucleotide, wherein the oligonucleotide comprises one or more modified pyrimidines, is capable of being cleaved by a CTC nuclease but is resistant to cleavage by non-CTC nucleases. In certain embodiments, the nuclease is an endonuclease. In certain embodiments, the nuclease is specific to CTCs. In certain embodiments, the nuclease is overexpressed in CTCs as compared to normal cells.

In certain embodiments, the oligonucleotide is 8-15 nucleotides in length. In certain embodiments, the oligonucleotide is 8-12 nucleotides in length. In certain embodiments, the oligonucleotide is 5′-CTACGTAG-3′ (SEQ ID NO:1), 5′-TCTCGTACGTAC-3′ (SEQ ID NO:2), 5′-CUACGAUG-3′ (SEQ ID NO:3) or 5′-UCUCGUACGUAC-3′ (SEQ ID NO:4). In certain embodiments, one or more of the nucleotides are chemically modified. In certain embodiments, one or more of the pyrimidines are chemically modified. In certain embodiments, one or more of the pyrimidines are 2′-O-methyl modified. In certain embodiments, one or more of the pyrimidines are 2′-fluoro modified. In certain embodiments, one or more of the purines are chemically modified. In certain embodiments, one or more of the purines are 2′-O-methyl modified. In certain embodiments, one or more of the purines are 2′-fluoro modified.

In certain embodiments, the fluorophore is selected from the group consisting of the fluorophores listed in Table 1, such as for example, a fluorophore that has an emission in the near infra-red range. In certain embodiments, the quencher is selected from the group consisting of the quenchers listed in Table 2.

In certain embodiments, the oligonucleotide is single-stranded.

In certain embodiments, the oligonucleotide comprises both RNA and DNA.

The present invention in certain embodiments further provides a method of detecting a circulating tumor cell (CTC) in a sample comprising measuring fluorescence of a sample that has been contacted with a substrate probe of any one of claims 1-16, wherein a fluorescence level that is greater than the fluorescence level of a control indicates that the sample has a CTC. In certain embodiments, the test fluorescence level is at least 1-100% greater than the control level. In certain embodiments, the fluorophore absorbs in the range of 650-850 nm. In certain embodiments, the CTC is a metastatic breast cancer cell.

In certain embodiments, the fluorophore is FAM, Cy5, Cy5.5, Cy7, Licor IRDye 700, Cy7.5, Dy780, Dy781, DyLight 800, Licor IRDye 800 CW, or Alexa Fluor 647, 660, 680, 750, or 790. In certain embodiments, the fluorophore is FAM or Cy5.5. In certain embodiments, the fluorophore is FAM, TET, HEX, JOE, MAX, Cy3, or TAMRA and the quencher is IBFQ, BHQ1, BHQ2, or Licor IRDye QC-1. In certain embodiments, the fluorophore is ROX, Texas Red, Cy5, or Cy5.5 and the quencher is IBRQ or BHQ2.

The present invention in certain embodiments further provides a method for detecting CTC nuclease activity in a test sample, comprising:

(a) Contacting the test sample with a composition comprising substrate probe described above, thereby creating a test reaction mixture,

(b) Incubating the test reaction mixture for a time sufficient for cleavage of the substrate probe by a CTC nuclease in the sample; and

(c) Determining whether a detectable fluorescence signal is emitted from the test reaction mixture, wherein emission of a fluorescence signal from the reaction mixture indicates that the sample contains CTC nuclease activity. In certain embodiments, the composition further comprises a buffer at pH8 to pH10. In certain embodiments, the pH is about pH9. In certain embodiments, the composition further comprises Mg²⁺ at a concentration of 2 mM to 20 mM. In certain embodiments, the Mg²⁺ is at a concentration of about 10 mM. In certain embodiments, the composition lacks Triton X-100.

The present invention in certain embodiments further provides a method for detecting CTC nuclease activity in a test sample, comprising:

(a) contacting the test sample with a composition comprising substrate probe as described above, thereby creating a test reaction mixture,

(b) Incubating the test reaction mixture for a time sufficient for cleavage of the substrate probe by a CTC nuclease in the sample; and

(c) Determining whether a detectable fluorescence signal is emitted from the test reaction mixture;

(d) Contacting a control sample with the substrate probe, wherein the control sample comprises a predetermined amount of nuclease, thereby creating a control reaction mixture;

(e) Incubating the control reaction mixture for a time sufficient for cleavage of the substrate probe by a nuclease in the control sample; and

(f) determining whether a detectable fluorescence signal is emitted from the control reaction mixture; wherein detection of a greater fluorescence signal in the test reaction mixture than in the control reaction mixture indicates that the test sample contains greater nuclease activity than in the control sample, and wherein detection of a lesser fluorescence signal in the test reaction mixture than in the control reaction mixture indicates that the test sample contains less nuclease activity than in the control sample.

In certain embodiments, the predetermined amount of nuclease is no nuclease, such that detection of a greater fluorescence signal in the test reaction mixture than in the control reaction mixture indicates that the test sample contains nuclease activity. In certain embodiments, the method further comprises contacting the test sample with a buffer before or during step (a). In certain embodiments, the control comprises K562 cells. In certain embodiments, the method can detect CTCs in a sample containing fewer than 10 CTCs/sample. In certain embodiments, the substrate probe is present at a concentration of about 6.25 pmol.

As used herein, the term “about” means±10%.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides

“Operably-linked” refers to the association two chemical moieties so that the function of one is affected by the other, e.g., an arrangement of elements wherein the components so described are configured so as to perform their usual function.

### BRIEF DESCRIPTION OF DRAWINGS

FIG. 1. Survival curves for patients with breast cancer. Upper line <5 CTCs per 7.5 mL of blood (Favorable). Lower line ≥5 CTCs per 7.5 mL of blood (Unfavorable). (Miller et al., Journal of Oncology, Volume 2010, Article ID 617421). High levels of CTCs correlate with lower survival.

FIG. 2. Four oligonucleotide probes designed to test for activation by lysates from breast cancer cell lines. Two DNA probes (Oligo 1 and DNA probe) and two RNA probe (Self-Hyb F1 and 2′ F1 Pyr). The RNA probes are comprised of 2′-fluro-modified pyrimidines and canonical purines. Each probe has a 5′ fluorophore (FAM), represented by the green stars, and a 3′ quencher (ZEN), represented by the black circles.

FIG. 3. An oligonucleotide, labeled with a fluorophore, is not fluorescent due to the close proximity of a quencher on the opposite end. Upon degradation of the oligo, the quencher diffuses away from the fluorophore and the fluorophore exhibits fluorescence (from Hernandez, et al., Nucleic Acid Ther. 2012 February; 22(1):58-68).

FIG. 4. Nucleases present in lysates from breast cancer cell lines activate oligonucleotide probes.

FIG. 5. Nucleases present in conditioned media of breast cancer cell lines activate oligonucleotide probes.

FIG. 6. Optimization of nuclease activation buffer pH. Lysates from MDA231 cells, human PBMC cells (a), or human serum (b) were dialyzed with Tris-buffer ranging from pH 7 to pH 10. Nuclease activity was highest at pH9.

FIG. 7: Optimization of cation concentration in nuclease activation buffer. Lysates from MDA231 cell lysates were dialyzed with buffer containing various concentrations of Ca²⁺ or Mg²⁺. 10 mM Mg²⁺ resulted in highest nuclease activity in and Ca²⁺ concentration had no effect.

FIG. 8: Kinetics of nuclease degradation of various probes (A=DNA Probe, B=2′F1 RNA Probe, C=Self-Hyb RNA Probe, D=Oligo 1 Probe) by a breast cancer cell line (MDA453), a lymphoblastic cell line (K562), or lysis buffer alone. Probes were digested 6 hours at 37° C., with measurements taken every 10 minutes.

FIGS. 9A-C: Buffer optimizations for nuclease activity assay. A) Effect of magnesium cation or calcium cation concentration and pH. B) Different amounts (A=100 pmol, B=25 pmol, C=12.5 pmol, D=6.25 pmol) of ssDNA probe were used in the nuclease activity assay for lysates from different numbers of SkBR3 cells (0, 10, 30, 100). C) Lysates from 100 Iowa 1T cells (black) or lysis buffer (gray) was mixed with 5, 2.5, 1, or 0.5 pmols of dsDNA, ssDNA, or 2′F-RNA probe, and incubated for 6 hours at 37° C.

FIG. 10. Probe sensitivity for breast cancer cells. Each of the probes was incubated with lysates derived from different amounts of Iowa1T breast cancer cells at 37° C. for 6 hrs. Fluorescence was measured in a microplate reader every 20 min.

FIGS. 11A-C: Probe selectivity for breast cancer cells. Probes were incubated with lysates of 100 cells from a lymphoblastic cell line (K562) or various breast cancer cell lines (BT20, MDA468, HCC1937, MDA231, MDA453, Iowa1T, MCF7, SKBr3, and BT474). Fold activation is defined as fluorescence produced by breast cancer cell lines divided by that produced by K562 cells. A) dsDNA Probe. B) ssDNA Probe C) 2′F-RNA Probe. Black bars are triple negative breast cancer cell lines, and white bars are hormone positive breast cancer cell lines.

FIG. 12: Whole blood from a human donor was spiked with 10⁵, 10⁴, 10³, or 0 SKBr3 breast cancer cells per 100 uL of blood. The blood with or without SKBr3 cells was pelleted at 700×g for 5 min, and supernatant was removed. The pellet was lysed with 100 uL of nuclease activation buffer, and the resulting lysates were used to digest Oligo 1, DNA probe, Self Hyb probe, or 2′F1 probe at 32 degrees for 3 hours.

FIG. 13: Iowa1T breast cancer cells were spiked into whole blood from a human donor and isolated with ISET filtration. The cancer cells were lysed on the ISET membrane and nuclease activity was measured after incubation at 6 hours at 37° C. Human blood with was spiked 500 (B+500), 150 (B+150), 50 (B+50), 15 (B+15), or 0 (B) Iowa1T cells per 0.33 mL of blood.

FIG. 14. Effect of external addition of cancer cells into healthy donor blood samples prior to ScreenCell CTC capture. Plotted are the signal intensities using the dsDNA probe in the nuclease activity assay. MDA453 breast cancer cells were added to blood samples of 0.1 mL prior to work up with the ScreanCell filter system.

FIGS. 15A-C. Evaluation of ISET and ScreenCell systems. A) ISET and ScreenCell filters were evaluated for their ability to remove blood cells from blood. Each of the probes was incubated with lysates derived from breast cancer cell line MDA-453 or lymphoblast cell line K562 at 37° C. for 6 hrs. Fluorscence was measured in a microplate reader every 15 min. Fluorescence intensity for each filter represents the background fluorescence of nucleases due to the retention blood cells on the filter. B) Average nuclease activity of all healthy donors when their blood is processed with the ScreenCell filters. C) Variability of 3 different healthy donors between blood draws performed on different days. Each graph represents a different donor with the dsDNA probe.

FIGS. 16A-B. Comparison of Healthy donor samples to CTC containing samples and stage IV breast cancer patient samples. A) Healthy donor sample activities with and without additionally spiked in breast cancer cells (200 Iowa1T cells). B) Stage IV breast cancer samples (gray circles) examined by dsDNA probe fluorescence compared to mean intensity from healthy donor samples (black triangles). Plotted are mean values and corresponding SEM for patients (n=28) and healthy donors (n=15). Asterisks indicate statistical significance (p-values of a 2-way ANOVA t-test are described in the depicted table).

FIG. 17. Variation of nuclease activity from identical donor samples. Blood collection dates can be obtained from FIG. 18.

FIG. 18. Blood cell counts from breast cancer patient and healthy donors. Patients blood showed a reduced number of all blood cells types in comparison to helthy donor blood. Grey shaded areas present the reference range considered as healthy.

FIGS. 19A-B: Serum-free media (8 mL OptiMEM) was conditioned by annotated cell lines (basal breast cancer, at ˜70% confluency) for 24 hrs. Subsequently media was collected and any residual cell debris present in the conditioned media removed by centrifugation. The conditioned media was concentrated and buffer was exchanged to optimize nucleases activity. For nuclease activity detection, two different nucleic acid probes (230 nM) were used, each bearing a fluorophore and an appropriate fluorophore-quencher. Shown are fluorescence signals over a time course of 6 hrs, (a) single stranded DNA(ssDNA)-probe (b) single stranded RNA (ssRNA)-probe (chemically modified).

FIGS. 19C-D: Serum-free media (8 mL OptiMEM) was conditioned by annotated cell lines (luminal breast cancer, at ˜70% confluency) for 24 hrs. Subsequently media was collected and any residual cell debris present in the conditioned media removed by centrifugation. The conditioned media was concentrated and buffer was exchanged to optimize nucleases activity. For nuclease activity detection, two different nucleic acid probes (230 nM) were used, each bearing a fluorophore and an appropriate fluorophore-quencher. Shown are fluorescence signals over a time course of 6 hrs, (c) single stranded DNA(ssDNA)-probe (d) single stranded RNA (snRNA)-probe (chemically modified).

FIGS. 20A-C: Serum-free media (8 mL OptiMEM) was conditioned by annotated cell lines (pancreatic cancer, at ˜70% confluency) for 24 hrs. Subsequently media was collected and any residual cell debris present in the conditioned media removed by centrifugation. The conditioned media was concentrated and buffer was exchanged to optimize nucleases activity. For nuclease activity detection, two different nucleic acid probes (230 nM) were used, each bearing a fluorophore and an appropriate fluorophore-quencher. Shown are fluorescence signals over a time course of 6 hrs, (a) double-stranded DNA (dsDNA)-probe, (b) single stranded DNA(ssDNA)-probe (c) single stranded RNA (ssRNA)-probe (chemically modified).

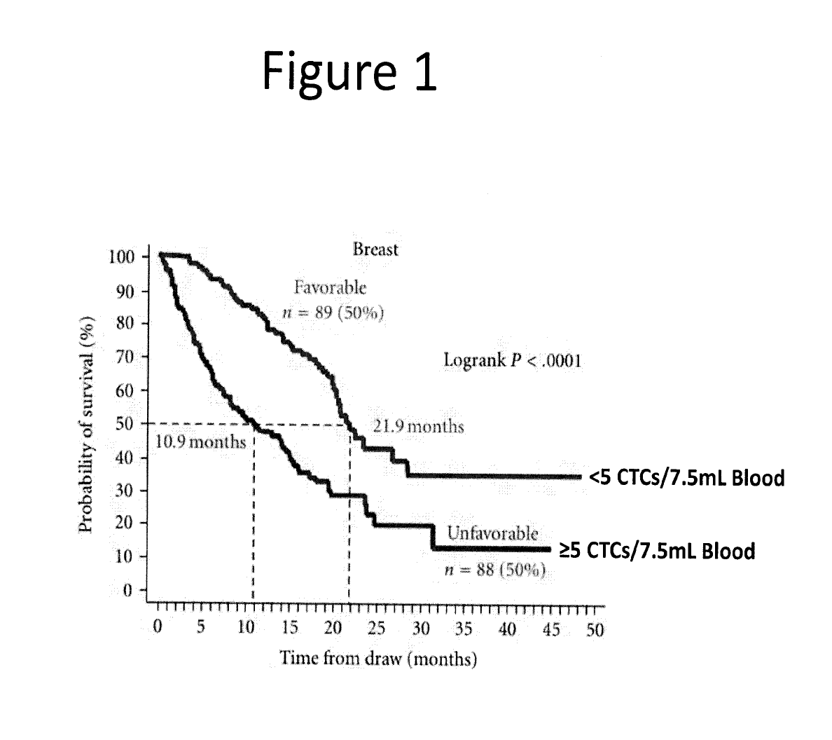

### DETAILED DESCRIPTION OF THE INVENTION

Novel diagnostic methods for characterizing the cellular and molecular makeup of metastatic breast cancer on an individualized basis (i.e., personalized medicine) have been intensively pursued in recent years due to the heterogeneity of this disease. The number of circulating tumor cells (CTCs) in cancer patients has recently been shown to be a valuable (and noninvasively accessible) diagnostic indicator of the state of metastatic breast cancer. In particular, patients with no CTCs were found to have a better overall prognosis compared to CTC-positive patients. However, the accuracy and ease-of-operation of available CTC tests remains unsatisfactory. The present invention provides a rapid and highly-sensitive CTC detection assay based on the development of chemically-modified, nuclease-activated probes that are specifically digested (i.e., activated) by target nucleases expressed in breast cancer cells that is straightforward to implement in most clinical diagnostic labs.

The present invention describes a diagnostic test kit for better staging of breast cancer and for detection of possible metastases. Current tests in the market are expensive, have high false positives and negatives, have high background noise, are time consuming and require a significant level of expertise to conduct.

The inventors have identified several nuclease probes that are digested by nucleases found in human breast cancer cell lines. The inventors have optimized conditions (buffer components, amount of probe, duration of assay, etc.) to increase the sensitivity of these probes. The inventors have demonstrated that the probes can detect as few as 10-30 cancer cells. The inventors have also been able to demonstrate specificity. For example the probes are not digested by lymphoblasts (e.g. K-562 cell line).

Chemical moieties that quench fluorescent light operate through a variety of mechanisms, including fluorescence resonance energy transfer (FRET) processes and ground state quenching. FRET is one of the most common mechanisms of fluorescent quenching and can occur when the emission spectrum of the fluorescent donor overlaps the absorbance spectrum of the quencher and when the donor and quencher are within a sufficient distance known as the Forster distance. The energy absorbed by a quencher can subsequently be released through a variety of mechanisms depending upon the chemical nature of the quencher. Captured energy can be released through fluorescence or through nonfluorescent mechanisms, including charge transfer and collisional mechanisms, or a combination of such mechanisms. When a quencher releases captured energy through nonfluorescent mechanisms FRET is simply observed as a reduction in the fluorescent emission of the fluorescent donor.

Although FRET is the most common mechanism for quenching, any combination of molecular orientation and spectral coincidence that results in quenching is a useful mechanism for quenching by the compounds of the present invention. For example, ground-state quenching can occur in the absence of spectral overlap if the fluorophore and quencher are sufficiently close together to form a ground state complex.

Quenching processes that rely on the interaction of two dyes as their spatial relationship changes can be used conveniently to detect and/or identify nucleotide sequences and other biological phenomena. As noted previously, the energy transfer process requires overlap between the emission spectrum of the fluorescent donor and the absorbance spectrum of the quencher. This complicates the design of probes because not all potential quencher/donor pairs can be used. For example, the quencher BHQ-1, which maximally absorbs light in the wavelength range of about 500-550 nm, can quench the fluorescent light emitted from the fluorophore fluorescein, which has a wavelength of about 520 nm. In contrast, the quencher BHQ-3, which maximally absorbs light in the wavelength range of about 650-700 nm would be less effective at quenching the fluorescence of fluorescein but would be quite effective at quenching the fluorescence of the fluorophore Cy5 which fluoresces at about 670 nm. The use of varied quenchers complicates assay development because the purification of a given probe can vary greatly depending on the nature of the quencher attached.

Many quenchers emit energy through fluorescence reducing the signal to noise ratio of the probes that contain them and the sensitivity of assays that utilize them. Such quenchers interfere with the use of fluorophores that fluoresce at similar wavelength ranges. This limits the number of fluorophores that can be used with such quenchers thereby limiting their usefulness for multiplexed assays which rely on the use of distinct fluorophores in distinct probes that all contain a single quencher.

Endonucleases are enzymes that cleave the phosphodiester bond within a polynucleotide (DNA or RNA) chain, in contrast to exonucleases, which cleave phosphodiester bonds at the end of a polynucleotide chain. Typically, a restriction site, i.e., a recognition site for an endonuclease, is a palindromic sequence four to six nucleotides long.

Substrate Probes

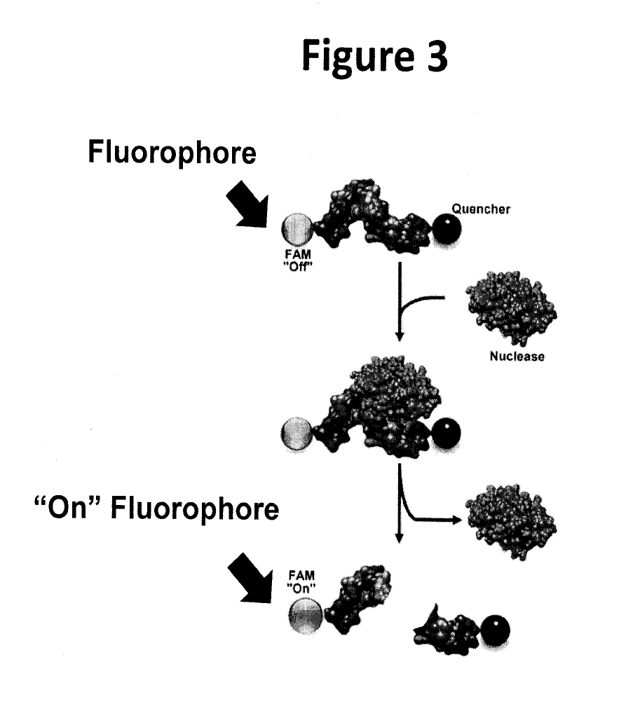

Hernandez et al. developed nuclease-activatable oligonucleotide probes to detect bacterial infections in vivo (FIG. 3). These probes provided a rapid, specific, and inexpensive method for detection of bacterial infection (Hernandez et al., Nucleic Acid Ther. 2012 February; 22(1):58-68).

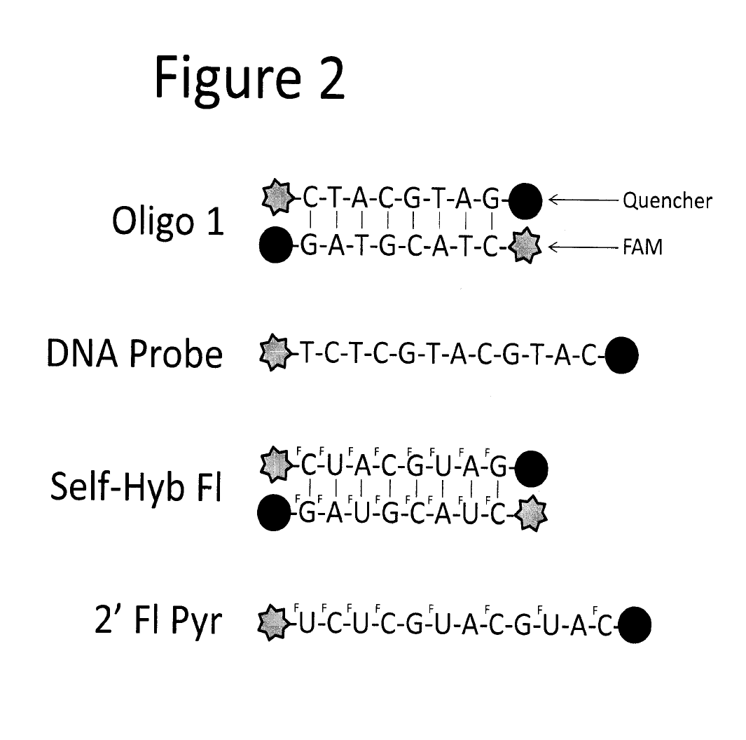

The present technology relates to a diagnostic test to detect circulating tumor cells from metastatic breast cancer in a patient's blood sample. The inventors have developed a method of detecting this with a variety of nuclease-activated oligonucleotide probes (FIG. 2), each of which is digested by nucleases found in human breast cancer cell lines. Preliminary tests have been conducted showing breast cancer cell lysates have high nuclease activity against the probes, the cells secrete nucleases, and the optimal conditions (pH and Mg²⁺ buffer concentration) for nuclease activity. It was possible to detect as few as 10-30 SKBr3 cells.

In certain embodiments, the present invention provides short oligonucleotide probes (substrate probes) composed of chemically modified RNA flanked with a fluorophore on one end and a fluorescence quencher on the other end. Upon cleavage of the probes by nucleases (e.g., ribonuclease), the fluorophore diffuses away from the quencher and exhibits fluorescence.

The present invention relates to methods for detecting nuclease (e.g., ribonuclease) activity in a sample, wherein the Substrate probe(s) comprises a single-stranded nucleic acid molecule containing at least one ribonucleotide or deoxyribonucleotide residue at an internal position that functions as a nuclease (e.g., ribonuclease) cleavage site (and in certain embodiments a 2′-fluoro modified pyrimidine or 2′-O-methyl modified pyrimidine that renders the oligonucleotide resistant to degradation by mammalian nucleases), a fluorescence reporter group on one side of the cleavage sites, and a fluorescence-quenching group on the other side of the cleavage site, and 2) visual detection of a fluorescence signal, wherein detection of a fluorescence signal indicates that a nuclease (e.g., ribonuclease) cleavage event has occurred, and, therefore, the sample contains nuclease (e.g., ribonuclease) activity. The compositions of the invention are also compatible with other detection modalities (e.g., fluorometry).

The substrate probe oligonucleotide of the invention comprises a fluorescent reporter group and a quencher group in such physical proximity that the fluorescence signal from the reporter group is suppressed by the quencher group. Cleavage of the substrate probe with a nuclease (e.g., ribonuclease) enzyme leads to strand cleavage and physical separation of the reporter group from the quencher group. Separation of reporter and quencher eliminates quenching, resulting in an increase in fluorescence emission from the reporter group. When the quencher is a so-called “dark quencher”, the resulting fluorescence signal can be detected by direct visual inspection (provided the emitted light includes visible wavelengths). Cleavage of the substrate probe compositions described in the present invention can also be detected by fluorometry.

In one embodiment, the synthetic substrate probe is an oligonucleotide comprising ribonucleotide residues. The synthetic substrate probe can also be a chimeric oligonucleotide comprising RNase-cleavable, e.g., RNA, residues, or modified RNase-resistant RNA residues. Substrate probe composition is such that cleavage is a ribonuclease-specific event and that cleavage by enzymes that are strictly deoxyribonucleases does not occur.

In one embodiment, the synthetic substrate probe is a chimeric oligonucleotide comprising ribonucleotide residue(s) and modified ribonucleotide residue(s). In one embodiment, the synthetic substrate probe is a chimeric oligonucleotide comprising ribonucleotide residues and 2′-O-methyl ribonucleotide residues. In one embodiment, the synthetic substrate probe is a chimeric oligonucleotide comprising 2′-O-methyl ribonucleotide residues and one or more of each of the four ribonucleotide residues, adenosine, cytosine, guanosine, and uridine. Inclusion of the four distinct ribonucleotide bases in a single substrate probe allows for detection of an increased spectrum of ribonuclease enzyme activities by a single substrate probe oligonucleotide.

In one embodiment, the synthetic substrate probe is an oligonucleotide comprising deoxyribonucleotide residues. The synthetic substrate probe can also be a chimeric oligonucleotide comprising DNase-cleavable, e.g., DNA, residues, or modified RNase-resistant RNA residues.

In one embodiment, the synthetic substrate probe is a chimeric oligonucleotide comprising deoxyribonucleotide residue(s) and modified ribonucleotide residue(s). In one embodiment, the synthetic substrate probe is a chimeric oligonucleotide comprising deoxyribonucleotide residues and 2′-O-methyl ribonucleotide residues. In one embodiment, the synthetic substrate probe is a chimeric oligonucleotide comprising 2′-O-methyl ribonucleotide residues and one or more of each of the four deoxyribonucleotide residues, deoxyadenosine, deoxycytosine, deoxyguanosine, and deoxythymidine. Inclusion of the four distinct deoxyribonucleotide bases in a single substrate probe allows for detection of an increased spectrum of deoxyribonuclease enzyme activities by a single substrate probe oligonucleotide.

To enable visual detection methods, the quenching group is itself not capable of fluorescence emission, being a “dark quencher”. Use of a “dark quencher” eliminates the background fluorescence of the intact substrate probe that would otherwise occur as a result of energy transfer from the reporter fluorophore. In one embodiment, the fluorescence quencher comprises dabcyl (4-(4′-dimethylaminophenylazo)benzoic acid). In one embodiment, the fluorescence quencher is comprised of QSY™-7 carboxylic acid, succinimidyl ester (N,N′-dimethyl-N,N′-diphenyl-4-((5-t-butoxycarbonylaminopentyl)aminocarbonyl) piperidinylsulfonerhodamine; a diarylrhodamine derivative from Molecular Probes, Eugene, Oreg.). Any suitable fluorophore may be used as reporter provided its spectral properties are favorable for use with the chosen quencher. A variety of fluorophores can be used as reporters, including but not limited to, fluorescein, tetrachlorofluorescein, hexachlorofluorescein, rhodamine, tetramethylrhodamine, Cy-dyes, Texas Red, Bodipy dyes, and Alexa dyes.

The method of the invention proceeds in two steps. First, the test sample is mixed with the substrate probe reagent and incubated. Substrate can be mixed alone with the test sample or will be mixed with an appropriate buffer, e.g., one of a composition as described herein. Second, visual detection of fluorescence is performed. As fluorescence above background indicates fluorescence emission of the reaction product, i.e. the cleaved substrate probe, detection of such fluorescence indicates that RNase activity is present in the test sample. The method provides that this step can be done with unassisted visual inspection. In particular, visual detection can be performed using a standard ultraviolet (UV) light source of the kind found in most molecular biology laboratories to provide fluorescence excitation. Substrate probes of the invention can also be utilized in assay formats in which detection of substrate probe cleavage is done using a multi-well fluorescence plate reader or a tube fluorometer.

The present invention further features kits for detecting nuclease (e.g., ribonuclease) activity comprising a substrate probe nucleic acid(s) and instructions for use. Such kits may optionally contain one or more of: a positive control nuclease (e.g., ribonuclease), RNase-free water, and a buffer. It is also provided that the kits may include RNase-free laboratory plasticware, for example, thin-walled, UV transparent microtubes for use with the visual detection method and/or multiwell plates for use with plate-fluorometer detection methods in a high-throughput format.

Accordingly, the present invention provides a method for detecting nuclease (e.g., ribonuclease) activity in a test sample, comprising: (a) contacting the test sample with a substrate probe, thereby creating a test reaction mixture, wherein the substrate probe comprises a nucleic acid molecule comprising (i) a cleavage domain comprising a single-stranded region, the single-stranded region comprising at least one internucleotide linkage (and in certain embodiments a 2′-fluoro modified pyrimidine or 2′-O-methyl modified pyrimidine that renders the oligonucleotide resistant to degradation by mammalian nucleases); (ii) a fluorescence reporter group on one side of the internucleotide linkage; and (iii) a non-fluorescent fluorescence-quenching group on the other side of the internucleotide linkage; (b) incubating the test reaction mixture for a time sufficient for cleavage of the substrate probe by a nuclease in the sample; and (c) determining whether a detectable fluorescence signal is emitted from the test reaction mixture, wherein emission of a fluorescence signal from the reaction mixture indicates that the sample contains nuclease activity.

While the methods of the invention can be practiced without the use of a control sample, in certain embodiments of the invention it is desirable to assay in parallel with the test sample a control sample comprising a known amount of nuclease activity. Where the control sample is used as a negative control, the control sample, in some embodiments, contains no detectable nuclease activity. Thus, the present invention further provides a method for detecting nuclease activity in a test sample, comprising: (a) contacting the test sample with a substrate probe, thereby creating a test reaction mixture, wherein the substrate probe comprises a nucleic acid molecule comprising: (i) a cleavage domain comprising a single-stranded region, the single-stranded region comprising at least one internucleotide linkage (and in certain embodiments a 2′-fluoro modified pyrimidine or 2′-O-methyl modified pyrimidine that renders the oligonucleotide resistant to degradation by mammalian nucleases); (ii) a fluorescence reporter group on one side of the internucleotide linkage; and (iii) a non-fluorescent fluorescence-quenching group on the other side of the internucleotide linkage; (b) incubating the test reaction mixture for a time sufficient for cleavage of the substrate probe by a nuclease (e.g., ribonuclease) activity in the test sample; (c) determining whether a detectable fluorescence signal is emitted from the test reaction mixture; (d) contacting a control sample with the substrate probe, the control sample comprising a predetermined amount of nuclease (e.g., ribonuclease), thereby creating a control reaction mixture; (e) incubating the control reaction mixture for a time sufficient for cleavage of the substrate probe by a nuclease (e.g., ribonuclease) in the control sample; (f) determining whether a detectable fluorescence signal is emitted from the control reaction mixture; wherein detection of a greater fluorescence signal in the test reaction mixture than in the control reaction mixture indicates that the test sample contains greater nuclease (e.g., ribonuclease) activity than in the control sample. In one embodiment, the predetermined amount of nuclease (e.g., ribonuclease) is no nuclease, such that detection of a greater fluorescence signal in the test reaction mixture than in the control reaction mixture indicates that the test sample contains nuclease (e.g., ribonuclease) activity.

The methods of the invention can further entail contacting the test sample with a buffer before or during step (a).

The present invention further provides compositions and kits for practicing the present methods. Thus, in certain embodiments, the present invention provides a nucleic acid comprising: (a) a cleavage domain comprising a single-stranded region, the single-stranded region comprising at least one internucleotide linkage (and in certain embodiments a 2′-fluoro modified pyrimidine or 2′-O-methyl modified pyrimidine that renders the oligonucleotide resistant to degradation by mammalian nucleases); (b) a fluorescence reporter group on one side of the internucleotide linkage; and (c) a non-fluorescent fluorescence-quenching group on the other side of the internucleotide linkage. In other embodiments, the present invention provides a kit comprising: (d) in one container, a substrate probe, the substrate probe comprising a nucleic acid molecule comprising a single stranded region, the single-stranded region comprising: (i) a cleavage domain comprising a single-stranded region, the single-stranded region comprising at least one internucleotide linkage 3′ to an adenosine residue, at least one internucleotide linkage 3′ to a cytosine residue, at least one internucleotide linkage 3′ to a guanosine residue, and at least one internucleotide linkage 3′ to a uridine residue, and wherein the cleavage domain does not comprise a deoxyribonuclease-cleavable internucleotide linkage; (ii) a fluorescence reporter group on one side of the internucleotide linkages; and (iii) a non-fluorescent fluorescence-quenching group on the other side of the internucleotide linkages.

In one embodiment of the foregoing methods and compositions, the single stranded region of the cleavage domain comprises at least one internucleotide linkage 3′ to an adenosine residue, at least one internucleotide linkage 3′ to a cytosine residue, at least one internucleotide linkage 3′ to a guanosine residue, and at least one internucleotide linkage 3′ to a uridine residue. In one embodiment, the cleavage domain does not comprise a deoxyribonuclease-cleavable internucleotide linkage. In yet another referred embodiment, the single stranded region of the cleavage domain comprises at least on internucleotide linkage 3′ to an adenosine residue, at least one internucleotide linkage 3′ to a cytosine residue, at least one internucleotide linkage 3′ to a guanosine residue, and at least one internucleotide linkage 3′ to a uridine residue and the cleavage domain does not comprise a deoxyribonuclease-cleavable internucleotide linkage.

In one embodiment of the foregoing methods and compositions, the single stranded region of the cleavage domain comprises at least one internucleotide linkage 3′ to a deoxyadenosine residue, at least one internucleotide linkage 3′ to a deoxycytosine residue, at least one internucleotide linkage 3′ to a deoxyguanosine residue, and at least one internucleotide linkage 3′ to a deoxythymidine residue. In one embodiment, the cleavage domain does not comprise a ribonuclease-cleavable internucleotide linkage. In yet another referred embodiment, the single stranded region of the cleavage domain comprises at least one internucleotide linkage 3′ to a deoxyadenosine residue, at least one internucleotide linkage 3′ to a deoxycytosine residue, at least one internucleotide linkage 3′ to a deoxyguanosine residue, and at least one internucleotide linkage 3′ to a deoxythymidine residue and the cleavage domain does not comprise a ribonuclease-cleavable internucleotide linkage.

With respect to the fluorescence quenching group, any compound that is a dark quencher can be used in the methods and compositions of the invention. Numerous compounds are capable of fluorescence quenching, many of which are not themselves fluorescent (i.e., are dark quenchers.) In one embodiment, the fluorescence-quenching group is a nitrogen-substituted xanthene compound, a substituted 4-(phenyldiazenyl)phenylamine compound, or a substituted 4-(phenyldiazenyl)naphthylamine compound. In certain specific modes of the embodiment, the fluorescence-quenching group is 4-(4′-dimethylaminophenylazo)benzoic acid), N,N′-dimethyl-N,N′-diphenyl-4-((5-t-butoxycarbonylaminopentyl) aminocarbonyl) piperidinylsulfonerhodamine (sold as QSY-7™ by Molecular Probes, Eugene, Oreg.), 4′,5′-dinitrofluorescein, pipecolic acid amide (sold as QSY-33™ by Molecular Probes, Eugene, Oreg.) 4-[4-nitrophenyldiazinyl]phenylamine, or 4-[4-nitrophenyldiazinyl]naphthylamine (sold by Epoch Biosciences, Bothell, Wash.). In other specific modes of the embodiment, the fluorescence-quenching group is Black-Hole Quenchers™ 1, 2, or 3 (Biosearch Technologies, Inc.).

In certain embodiments, the fluorescence reporter group is fluorescein, tetrachlorofluorescein, hexachlorofluorescein, rhodamine, tetramethylrhodamine, a Cy dye, Texas Red, a Bodipy dye, or an Alexa dye.

With respect to the foregoing methods and compositions, the fluorescence reporter group or the fluorescence quenching group can be, but is not necessarily, attached to the 5′-terminal nucleotide of the substrate probe.

The nucleic acids of the invention, including those for use as substrate probes in the methods of the invention, in certain embodiments are single-stranded RNA molecule. In other embodiments, the nucleic acids of the invention are chimeric oligonucleotides comprising a nuclease resistant modified ribonucleotide residue. Exemplary RNase resistant modified ribonucleotide residues include 2′-O-methyl ribonucleotides, 2′-methoxyethoxy ribonucleotides, 2′-O-allyl ribonucleotides, 2′-O-pentyl ribonucleotides, and 2′-O-butyl ribonucleotides. In one mode of the embodiment, the modified ribonucleotide residue is at the 5′-terminus or the 3′-terminus of the cleavage domain. In yet other embodiments, the nucleic acids of the invention are chimeric oligonucleotides comprising a deoxyribonuclease resistant modified deoxyribonucleotide residue. In specific modes of the embodiments, the deoxyribonuclease resistant modified deoxyribonucleotide residue is a phosphotriester deoxyribonucleotide, a methylphosphonate deoxyribonucleotide, a phosphoramidate deoxyribonucleotide, a phosphorothioate deoxyribonucleotide, a phosphorodithioate deoxyribonucleotide, or a boranophosphate deoxyribonucleotide. In yet other embodiments of the invention, the nucleic acids of the invention comprise a ribonuclease-cleavable modified ribonucleotide residue.

The nucleic acids of the invention, including those for use as substrate probes in the methods of the invention, are at least 3 nucleotides in length, such as 5-30 nucleotides in length. In certain specific embodiments, the nucleic acids of the invention are 5-20, 5-15, 5-10, 7-20, 7-15 or 8-12 nucleotides in length.

In certain embodiments, the fluorescence-quenching group of the nucleic acids of the invention is 5′ to the cleavage domain and the fluorescence reporter group is 3′ to the cleavage domain. In a specific embodiment, the fluorescence-quenching group is at the 5′ terminus of the substrate probe. In another specific embodiment, the fluorescence reporter group is at the 3′ terminus of the substrate probe.

In certain embodiments, the fluorescence reporter group of the nucleic acids of the invention is 5′ to the cleavage domain and the fluorescence-quenching group is 3′ to the cleavage domain. In a specific embodiment, the fluorescence reporter group is at the 5′ terminus of the substrate probe. In another specific embodiment, the fluorescence-quenching group is at the 3′ terminus of the substrate probe.

In one embodiment of the invention, a nucleic acid of the invention comprising the formula: 5′-N₁-n-N₂-3′, wherein: (a) “N₁” represents zero to five 2′-modified ribonucleotide residues; (b) “N₂” represents one to five 2′-modified ribonucleotide residues; and (c) “n” represents one to ten, such as four to ten unmodified ribonucleotide residues. In a certain specific embodiment, “N₁” represents one to five 2′-modified ribonucleotide residues. In certain modes of the embodiment, the fluorescence-quenching group or the fluorescent reporter group is attached to the 5′-terminal 2′-modified ribonucleotide residue of N₁.

In the nucleic acids of the invention, including nucleic acids with the formula: 5′-N₁-n-N₂-3′, the fluorescence-quenching group can be 5′ to the cleavage domain and the fluorescence reporter group is 3′ to the cleavage domain; alternatively, the fluorescence reporter group is 5′ to the cleavage domain and the fluorescence-quenching group is 3′ to the cleavage domain.

With respect to the kits of the invention, in addition to comprising a nucleic acid of the invention, the kits can further comprise one or more of the following: a ribonuclease; ribonuclease-free water, a buffer, and ribonuclease-free laboratory plasticware.

Substrate Probe Oligonucleotides

Compositions of the invention comprise synthetic oligonucleotide substrate probes that are substrate probes for nuclease (e.g., ribonuclease) enzymes. Substrate oligonucleotides of the invention comprise: 1) one or more nuclease-cleavable bases, e.g., RNA bases, some or all of which function as scissile linkages, 2) a fluorescence-reporter group and a fluorescence-quencher group (in a combination and proximity that permits visual FRET-based fluorescence quenching detection methods), and 3) may optionally contain RNase-resistant modified RNA bases, nuclease-resistant DNA bases, or unmodified DNA bases. Synthetic oligonucleotide RNA-DNA chimeras wherein the internal RNA bonds function as a scissile linkage are described in U.S. Pat. Nos. 6,773,885 and 7,803,536. The fluorescence-reporter group and the fluorescence-quencher group are separated by at least one RNAse-cleavable residue, e.g., RNA base. Such residues serve as a cleavage domain for ribonucleases.

In certain embodiments, the substrate probe oligonucleotide probes are single-stranded or double-stranded oligoribonucleotides. In certain embodiments, the oligonucleotide probes are composed of modified oligoribonucleotides. The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2-azido-ribose, carbocyclic sugar analogues a-anomeric sugars; epimeric sugars such as arabinose, xyloses or lyxoses, pyranose sugars, furanose sugars, and sedoheptulose. In certain embodiments, the substrate probe includes, but is not limited to, 2′-O-methyl RNA, 2′-methoxyethoxy RNA, 2′-O-allyl RNA, 2′-O-pentyl RNA, and 2′-O-butyl RNA. In certain embodiments, the substrate probe is an RNA-2′-O-methyl RNA oligonucleotide having the general structure 5′ r-NnN-q 3′, where ‘N’ represents from about one to five 2′-modified ribonucleotide residues, ‘n’ represents one to ten unmodified ribonucleotide residues, ‘r’ represents a fluorescence reporter group, and ‘q’ represents a fluorescence quencher group. The 5′- and 3′-position of reporter and quencher are interchangeable. In one embodiment, the fluorescence reporter group and the fluorescence quencher group are positioned at or near opposing ends of the molecule. It is not important which group is placed at or near the 5′-end versus the 3′-end. It is not required that the reporter and quencher groups be end modifications, however positioning these groups at termini simplifies manufacture of the substrate probe. The fluorescence reporter group and the fluorescence quencher group may also be positioned internally so long as an RNA scissile linkage lies between reporter and quencher.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4,N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methyl cytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine.

The oligonucleotides of the invention are synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR2; P(O)R′; P(O)OR6; CO; or CONR′2 wherein R is H (or a salt) or alkyl (1-12C) and R6 is alkyl (1-9C) is joined to adjacent nucleotides through —O— or —S—.

In certain embodiments of the present invention, the oligonucleotides have additional modifications, such as 2′O-methyl modification of the pyrimidines. In other embodiments, all of the nucleotides in the oligonucleotides are 2′O-methyl modified. Alternatively, the pyrimidines, or all the nucleotides, may be modified with 2′fluoros (both pyrimidines and purines).

The oligonucleotides are short, such as between 2-30 nucleotides in length (or any value in between, i.e., 2, 3, 4, 5, 6, 7, 8, 9, 10, 11, 12, 13, 14, 15, 16, 17, 18, 19, 20, 21, 22, 23, 24, 25, 26, 27, 28, 29, or 30). In certain embodiments, that oligonucleotide is between 10-15 nucleotides in length. In certain embodiments, that oligonucleotide is between 11-13 nucleotides in length. In general, shorter sequences will give better signal to noise ratios than longer probes and will therefore be more sensitive. However, in certain embodiments, shorter probes might not be the best substrate probe for the nuclease, so some degree of empiric optimization for length is needed. In certain embodiments, the oligonucleotide comprises 0-50% purines (or any value in between). In certain embodiments the oligonucleotide comprises 100% pyrimidines.

It should be noted that the specific sequence of the oligonucleotide is not critical. Endonucleases are enzymes that cleave the phosphodiester bond within a polynucleotide chain, in contrast to exonucleases, which cleave phosphodiester bonds at the end of a polynucleotide chain. Some endonucleases cleave single-stranded nucleic acid molecules, while others cleave double-stranded nucleic acid molecules. For example, the data below show a time-course of activity of the mycoplasma-derived nuclease and demonstrate that the mycoplasma nuclease can digest a variety of distinct sequences. The earliest time-point shows partial degradation of the 51 nt long sequence modified with either 2′-fluoro or 2′-O-methyl pyrimidines, with intermediate degradation products clearly visible. Each of the degradation products of intermediate size is in fact a distinct substrate probe and these are clearly being digested as seen in the later time points.

Fluorophores

In certain embodiments, the oligonucleotides of the present invention are operably linked to one or more fluorophores, which may also be called a “fluorescent tag.” A fluorophore is a molecule that absorbs light (i.e., excites) at a characteristic wavelength and emits light (i.e., fluoresces) at a second lower-energy wavelength. Fluorescence reporter groups that can be incorporated into substrate probe compositions include, but are not limited to, fluorescein, tetrachlorofluorescein, hexachlorofluorescein, tetramethylrhodamine, rhodamine, cyanine-derivative dyes, Texas Red, Bodipy, and Alexa dyes. Characteristic absorption and emission wavelengths for each of these are well known to those of skill in the art.

A fluorescence quencher is a molecule that absorbs or releases energy from an excited fluorophore (i.e., reporter), returning the fluorophore to a lower energy state without fluorescence emission at the wavelength characteristic of that fluorophore. For quenching to occur, reporter and quencher must be in physical proximity. When reporter and quencher are separated, energy absorbed by the reporter is no longer transferred to the quencher and is instead emitted as light at the wavelength characteristic of the reporter. Appearance of a fluorescent signal from the reporter group following removal of quenching is a detectable event and constitutes a “positive signal” in the assay of the present invention, and indicates the presence of nuclease in a sample.

Fluorescence quencher groups include molecules that do not emit any fluorescence signal (“dark quenchers”) as well as molecules that are themselves fluorophores (“fluorescent quenchers”). Substrate compositions that employ a “fluorescent quencher” will emit light both in the intact and cleaved states. In the intact state, energy captured by the reporter is transferred to the quencher via FRET and is emitted as light at a wavelength characteristic for the fluorescent quencher. In the cleaved state, energy captured by the reporter is emitted as light at a wavelength characteristic for the reporter. When compositions that employ fluorescent quenchers are used in a FRET assay, detection must be done using a fluorometer. In certain embodiments, substrate probe compositions that employ a “dark quencher” will emit light only in the cleaved state, enabling signal detection to be performed visually (detection may also be done using a fluorometer). Visual detection is rapid, convenient, and does not require the availability of any specialized equipment. It is desirable for an RNase detection assay to have visual detection method as an available option. Substrate probe compositions employing a “dark quencher” enable a visual detection nuclease assay while substrate probe compositions employing a “fluorescent quencher” are incompatible with a visual detection assay.

In one embodiment of the invention, the substrate probe is comprised of a fluorescence quencher group that does not itself emit a fluorescence signal, i.e. is a “dark quencher”. “Dark quenchers” useful in compositions of the invention include, but are not limited to, dabcyl, QSY™-7, QSY-33 (4′,5-dinitrofluorescein, pipecolic acid amide) and Black-Hole Quenchers™ 1, 2, and 3 (Biosearch Technologies, Novato, Calif.). Assay results (i.e., signal from cleaved substrate probe) can thus be detected visually. Optionally, the fluorescence signal can be detected using a fluorometer or any other device capable of detecting fluorescent light emission in a quantitative or qualitative fashion.

In certain embodiments, the fluorophore is one or more of the fluorophores listed in Table 1.

TABLE 1

| Probe | Excitation (nm) | Emission (nm) |
|---|---|---|
| Hydroxycoumarin | 325 | 386 |
| Alexa fluor | 325 | 442 |
| Aminocoumarin | 350 | 445 |
| Methoxycoumarin | 360 | 410 |
| Cascade Blue | (375); 401 | 423 |
| Pacific Blue | 403 | 455 |
| Pacific Orange | 403 | 551 |
| Lucifer yellow | 425 | 528 |
| Alexa fluor 430 | 430 | 545 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| Red 613 | 480; 565 | 613 |
| PerCP | 490 | 675 |
| Cy2 | 490 | 510 |
| TruRed | 490, 675 | 695 |
| FluorX | 494 | 520 |
| Fluorescein | 495 | 519 |
| FAM | 495 | 515 |
| BODIPY-FL | 503 | 512 |
| TET | 526 | 540 |
| Alexa fluor 532 | 530 | 555 |
| HEX | 535 | 555 |
| TRITC | 547 | 572 |
| Cy3 | 550 | 570 |
| TMR | 555 | 575 |
| Alexa fluor 546 | 556 | 573 |
| Alexa fluor 555 | 556 | 573 |
| Tamara | 565 | 580 |
| X-Rhodamine | 570 | 576 |
| Lissamine Rhodamine B | 570 | 590 |
| ROX | 575 | 605 |
| Alexa fluor 568 | 578 | 603 |
| Cy3.5 581 | 581 | 596 |
| Texas Red | 589 | 615 |
| Alexa fluor 594 | 590 | 617 |
| Alexa fluor 633 | 621 | 639 |
| LC red 640 | 625 | 640 |
| Allophycocyanin (APC) | 650 | 660 |
| Alexa fluor 633 | 650 | 688 |
| APC-Cy7 conjugates | 650; 755 | 767 |
| Cy5 | 650 | 670 |
| Alexa fluor 660 | 663 | 690 |
| Cy5.5 | 675 | 694 |
| LC red 705 | 680 | 710 |
| Alexa fluor 680 | 679 | 702 |
| Cy7 | 743 | 770 |
| IRDye 800 CW | 774 | 789 |

In certain in vivo embodiments, the fluorophore emits in the near infrared range, such as in the 650-900 nm range. (Weissleder et al., “Shedding light onto live molecular targets, Nature Medicine, 9:123-128 (2003)).

Fluorescence Quencher Group

In certain embodiments, the oligonucleotides of the present invention are operably linked to one or more fluorescence quencher group or “quencher.”

In certain embodiments, the quencher is one or more of the quenchers listed in Table 2.

TABLE 2

| Quencher | Absorption Maximum (nm) |
|---|---|
| DDQ-I | 430 |
| Dabcyl | 475 |
| Eclipse | 530 |
| Iowa Black FQ | 532 |
| BHQ-1 | 534 |
| QSY-7 | 571 |
| BHQ-2 | 580 |
| DDQ-II | 630 |
| Iowa Black RQ | 645 |
| QSY-21 | 660 |
| BHQ-3 | 670 |
| IRDye QC-1 | 737 |

Additional quenchers are described in U.S. Pat. No. 7,439,341, which is incorporated by reference herein.

Linkers

In certain embodiments, the oligonucleotide is linked to the fluorophore and/or quencher by means of a linker.

In certain embodiments, an aliphatic or ethylene glycol linker (as are well known to those will skill in the art) is used. In certain embodiments, the linker is a phosphodiester linkage. In certain embodiments, the linker is a phosphorothioate linkage. In certain embodiments, other modified linkages between the modifier groups like dyes and quencher and the bases are used in order to make these linkages more stabile, thereby limiting degradation to the nucleases.

In certain embodiments, the linker is a binding pair. In certain embodiments, the “binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-receptor, receptor-ligand, enzyme-substrate probe, IgG-protein A, antigen-antibody, and the like. In certain embodiments, a first member of the binding pair comprises avidin or streptavidin and a second member of the binding pair comprises biotin.

In certain embodiments, the oligonucleotide is linked to the fluorophore and/or quencher by means of a covalent bond.

In certain embodiments, the oligonucleotide probe, i.e., an oligonucleotide that is operably linked to a fluorophore and quencher, is also operably linked to a solid substrate. For example, the oligonucleotide probe may be linked to a magnetic bead.

Chemistries that can be used to link the fluorophores and quencher to the oligonucleotide are known in the art, such as disulfide linkages, amino linkages, covalent linkages, etc. In certain embodiments, aliphatic or ethylene glycol linkers that are well known to those with skill in the art can be used. In certain embodiments phosphodiester, phosphorothioate and/or other modified linkages between the modifier groups like dyes and quencher are used. These linkages provide stability to the probes, thereby limiting degradation to nucleobases. Additional linkages and modifications can be found on the world-wide-web at trilinkbiotech.com/products/oligo/oligo_modifications.asp.

Detection Compositions

In certain embodiments, the probes described above can be prepared as pharmaceutically-acceptable compositions. In certain embodiments, the probes are administered so as to result in the detection of a microbial infection. The amount administered will vary depending on various factors including, but not limited to, the composition chosen, the particular disease, the weight, the physical condition, and the age of the mammal. Such factors can be readily determined by the clinician employing animal models or other test systems, which are well known to the art.

Pharmaceutical formulations, dosages and routes of administration for nucleic acids are generally known in the art. The present invention envisions detecting a microbial infection in a mammal by the administration of a probe of the invention. Both local and systemic administration is contemplated.

One or more suitable unit dosage forms of the probe of the invention can be administered by a variety of routes including parenteral, including by intravenous and intramuscular routes, as well as by direct injection into the diseased tissue. The formulations may, where appropriate, be conveniently presented in discrete unit dosage forms and may be prepared by any of the methods well known to pharmacy. Such methods may include the step of bringing into association the probe with liquid carriers, solid matrices, semi-solid carriers, finely divided solid carriers or combinations thereof, and then, if necessary, introducing or shaping the product into the desired delivery system.

When the probes of the invention are prepared for administration, in certain embodiments they are combined with a pharmaceutically acceptable carrier, diluent or excipient to form a pharmaceutical formulation, or unit dosage form. The total active ingredient (i.e., probe) in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules, as a solution, a suspension or an emulsion.

Pharmaceutical formulations containing the probe of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of probe of the invention can also take the form of an aqueous or anhydrous solution or dispersion, or alternatively the form of an emulsion or suspension.

Thus, probe may be formulated for parenteral administration (e.g., by injection, for example, bolus injection or continuous infusion) and may be presented in unit dose form in ampules, pre-filled syringes, small volume infusion containers or in multi-dose containers with an added preservative. The probe may take such forms as suspensions, solutions, or emulsions in oily or aqueous vehicles, and may contain formulatory agents such as suspending, stabilizing and/or dispersing agents. Alternatively, the probe may be in powder form, obtained by aseptic isolation of sterile solid or by lyophilization from solution, for constitution with a suitable vehicle, e.g., sterile, pyrogen-free water, before use.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0, saline solutions, and water.

Substrate Probe Synthesis

Synthesis of the nucleic acid substrate probe of the invention can be performed using solid-phase phosphoramidite chemistry (U.S. Pat. No. 6,773,885) with automated synthesizers, although other methods of nucleic acid synthesis (e.g., the H-phosphonate method) may be used. Chemical synthesis of nucleic acids allows for the production of various forms of the nucleic acids with modified linkages, chimeric compositions, and nonstandard bases or modifying groups attached in chosen places throughout the nucleic acid's entire length.

Detection Methods

In certain embodiments, the present invention provides methods for detecting CTCs in a sample in vitro. The method of the invention proceeds in the following steps: combine “test sample” with substrate probe(s) to produce a mixture, the mixture being the Assay Mix, incubate, and detect fluorescence signal. “Test sample” refers to any material being assayed for nuclease activity and in certain embodiments, will be a liquid. Solids can be indirectly tested for the presence of RNase contamination by washing or immersion in solvent, e.g., water, followed by assay of the solvent.

Assay Mix

The substrate probe is mixed and incubated with the test sample. This mixture constitutes the Assay Mix. Ideally, the Assay Mix is a small volume, from about 1 μl to about 10 mls, or, from about 10 to 100 The precise volume of the Assay Mix will vary with the nature of the test sample and the detection method. Optionally, a buffer can be added to the Assay Mix. Nucleases, including some ribonucleases, require the presence of divalent cations for maximum activity and providing an optimized buffered solution can increase the reaction rate and thereby increase assay sensitivity. Buffers of different composition can be used, as described in U.S. Pat. No. 6,773,885. In certain embodiments, control reactions are included, but are not essential. A Negative Control Mix, for example, comprises a solution of substrate probe in water or buffer without any test sample or added nuclease. In this control, the substrate probe should remain intact (i.e., without fluorescence emission). If the Negative Control Mix results in positive signal, then the quality of all reagents is suspect and fresh reagents should be employed. Possible causes of a signal in a Negative Control include degradation of the substrate probe or contamination of any component reagent with nuclease activity. A Positive Control Mix, for example, comprises a solution of substrate probe in water or buffer plus a known, active RNase enzyme. If the Positive Control Mix results in a negative signal, then the quality of all reagents is suspect and fresh reagents should be employed. Possible causes of a negative Positive Control Mix include defective substrate probe or contamination of any component reagent with a nuclease inhibitor. Any RNase that cleaves the substrate probe can be employed for use in the Positive Control Mix. In one embodiment, RNase A is used, as this enzyme is both inexpensive and readily available. Alternatively, RNase 1 can be used. RNase 1 is heat labile and is more readily decontaminated from laboratory surfaces.

Incubation.

The Assay Mix (e.g., the test sample plus substrate probe) is incubated. Incubation time and condition can vary from a few minutes to 24 hours or longer depending upon the sensitivity required. Incubation times of one hour or less are desirable. Nucleases are catalytic. Increasing incubation time should therefore increase sensitivity of the Assay, provided that background cleavage of the substrate probe (hydrolysis) remains low. As is evident, assay background is stable over time and Assay sensitivity increases with time of incubation. Incubation temperature can generally vary from room temperature to 37° C. but may be adjusted to the temperature optimum of a specific nuclease suspected as being present as a contaminant.

Signal Detection.

Fluorescence emission can be detected using a number of techniques (U.S. Pat. No. 6,773,885). In one method of detection, visual inspection is utilized. Visual detection is rapid, simple, and can be done without need of any specialized equipment. Alternatively, detection can be done using fluorometry or any other method that allows for qualitative or quantitative assessment of fluorescent emission.

Visual Detection Method.

Following incubation, the Assay Mix is exposed to UV light to provide excitation of the fluorescence reporter group. An Assay Mix in which the substrate probe remains intact will not emit fluorescent signal and will visually appear clear or dark. Absence of fluorescence signal constitutes a negative assay result. An Assay Mix in which the substrate probe has been cleaved will emit fluorescent signal and will visually appear bright. Presence of fluorescence signal constitutes a positive assay result, and indicates the presence of RNase activity in the sample. The visual detection method is primarily intended for use as a qualitative nuclease assay, with results being simply either “positive” or “negative”. However, the assay is crudely quantitative in that a bright fluorescent signal indicates higher levels of RNase contamination than a weak fluorescent signal.

The Assay Mix will ideally constitute a relatively small volume, for example 10 to 100 μl, although greater or lesser volumes can be employed. Small volumes allow for maintaining high concentrations of substrate probe yet conserves use of substrate probe. The visual detection Assay in one embodiment uses 50 pmoles of substrate probe at a concentration of 0.5 μM in a 100 μl final volume Assay Mix. Lower concentration of substrate probe (e.g., below 0.1 uM) will decrease assay sensitivity. Higher concentrations of substrate probe (e.g., above 1 μM) will increase background and will unnecessarily consume substrate probe.

Steps (mixing, incubating, detecting), can be performed in one tube. In one embodiment, the tube is a small, thin-walled, UV transparent microfuge tube, although tubes of other configuration may be used. A “short wave” UV light source emitting at or around 254 nm is used in one embodiment for fluorescence excitation. A “long wave” UV light source emitting at or around 300 nm can also be employed. A high intensity, short wave UV light source will provide for best sensitivity. UV light sources of this kind are commonly found in most molecular biology laboratories. Visual detection can be performed at the laboratory bench or in the field, however sensitivity will be improved if done in the dark.

Fluorometric Detection Method.

Following incubation fluorescence emission can be detected using a fluorometer. Fluorometric detection equipment includes, but is not limited to, single sample cuvette devices and multiwell plate readers. As before, mixing, incubation, and detection can be performed in the same vessel. Use of a multiwell plate format allows for small sample volumes, such as 200 μl or less, and high-throughput robotic processing of many samples at once. This format is used in certain industrial QC settings. The method also provides for the Assay to be performed in RNase free cuvettes. As before, mixing, incubation, and detection can be performed in the same vessel. Use of fluorometric detection allows for highly sensitive and quantitative detection.

Kits

The present invention further includes kits for detecting nuclease activity in a sample, comprising substrate probe nucleic acid(s) and instructions for use. Such kits may optionally contain one or more of: a positive control nuclease, RNase-free water, a buffer, and other reagents. The kits may include RNase-free laboratory plasticware, such as thin-walled, UV transparent microtubes and/or multiwell plates for use with the visual detection method and multiwell plates for use with plate-fluorometer detection methods.

The assay is compatible with visual detection. In certain embodiments, the substrate probe will be provided in dry form in individual thin-walled, UV transparent microtubes, or in multiwell (e.g., 96 well) formats suitable for high throughput procedures. Lyophilized substrate probe has improved long-term stability compared to liquid solution in water or buffer. If provided in liquid solution, stability is improved with storage at least below −20° C., such as at −80° C. Storage in individual aliquots limits potential for contamination with environmental nucleases. Alternatively, the substrate probe can be provided in bulk, either lyophilized or in liquid solution. Alternatively, substrate probe can be provided in bulk and can be dispersed at the discretion of the user.

In Vitro Assays for Evaluating Nuclease Activity

In certain embodiments, the present invention provides in vitro assays for evaluating the activity of CTC nucleases on various nucleic acid substrate probes. For example, a biological sample (e.g., biological fluids) or material derived from such a sample is combined with an oligonucleotide-based probe and incubated for a period to time. The fluorescence level of this reaction is then measured (e.g., with a fluorometer), and compared with the fluorescence levels of similar reactions that serve as positive and negative controls.

### Example 1

### Rapid and Sensitive Detection of Circulating Tumor Cells with Nuclease-Activated Oligonucleotide Probes

Metastatic breast cancer is the second leading cause of female cancer deaths in the United States. Despite substantial progress in its treatment, metastatic breast cancer remains incurable. Early identification of breast cancer patients at greatest risk of developing metastatic disease is thus an important goal that would enable oncologists to aggressively treat these patients while the cancer is still vulnerable. In addition, this would spare patients who do not need or would not benefit from further treatments from having to endure the harmful side-effects of chemotherapeutic drug regimens. Circulating tumor cells (CTCs) are rare cancer cells found in the blood circulation of cancer patients that provide a non-invasively accessible cancer cell specimen (liquid biopsy) from patients. The number of circulating tumor cells (CTCs) in cancer patients has recently been shown to be a valuable diagnostic indicator of the state of metastatic breast cancer. In particular, patients with few or no CTCs were found to have a better overall prognosis compared to patients with high numbers of CTCs.

Despite the implications of CTCs as diagnostics for advanced breast cancer treatment, a critical challenge for adopting CTC-based diagnostic tests has been the development of methods with sufficient sensitivity to reliably detect the small number of CTCs that are present in the circulation. Furthermore, current tests for CTC detection are expensive, have high false positives and negatives, have high background noise, are time consuming and require a significant level of expertise to conduct. To overcome the limitations of current CTC detection assays and develop more sensitive, rapid and cost effective CTC detection methods, we explored the potential of detecting CTCs by measuring their nuclease activity with nuclease-activated probes (Hernandez F J, et al., Noninvasive imaging of staphylococcus aureus infections with a nuclease-activated probe. Nat Med. 2014; 20:301-306; Hernandez F J, et al., Degradation of nuclease-stabilized RNA oligonucleotides in mycoplasma-contaminated cell culture media. Nucleic Acid Ther. 2012; 22:58-68).

Data is presented toward the development of a rapid and highly-sensitive CTC detection assay based on nuclease-activated oligonucloetide probes that are selective digested (activated by target nucleases expressed in breast cancer cells. It was confirmed that these probes were not activated by serum nucleases or nucleases from a lymphoblastic cell line (e.g., K-562). Furthermore, we present extensive data towards the optimization of activity and sensitivity of these probes in cell lysates from various breast cancer cell lines and in blood from breast cancer patients. In conclusion, this work describes a robust assay for detection of breast cancer CTCs that is straightforward to implement in most clinical diagnostic labs.

Chemically Modified Oligonucleotides

Artificial RNA reagents such as siRNAs and aptamers often must be chemically modified for optimal effectiveness in environments that include nucleases. Synthetic RNA that is exposed to cells or tissues must be protected from nuclease degradation in order to carry out its intended function in most cases. Common approaches for avoiding nuclease degradation include nanoparticle encapsulation which insulates the RNA from exposure to nucleases and chemical modification to render it resistant to degradation. Modification of RNA by substituting O-methyl or fluoro groups for the hydroxyl at the 2′-position of the ribose can greatly enhance its stability in the presence of extracellular mammalian nucleases.

These modifications are widely employed in the development of siRNAs and RNA aptamers for both research and therapeutic applications. siRNAs can be modified with 2′-O-methyl substitutions in both sense and antisense strands without loss of silencing potency, but only a subset of nucleotides are typically modified with 2′-O-methyls as over-modification of the siRNA can reduce or eliminate its silencing ability. siRNAs with 2′-fluoro modified pyrimidines have also been reported to retain silencing activity in vitro as well as in vivo.

Substrates probes were synthesized with chemical modifications indicated in figure legends, flanked by a FAM (5′-modification) and a pair of fluorescence quenchers, “ZEN” and “Iowa Black” (3′-modifications).

Oligonucleotide Probe Synthesis and Purification

Oligonucleotide probes were synthesized and purified. Briefly, all the FAM-labeled probes were synthesized using standard solid phase phosphoramidite chemistry, followed by high performance liquid chromatography (HPLC) purification. For the Cy5.5-labeled probes, the sequences were first synthesized with ZEN and Iowa Black quenchers or inverted dT on the 3′-ends and amine on the 5′-ends using the standard solid phase phosphoramidite chemistry, and purified with HPLC. These purified sequences were then set to react with Cy5.5 NHS ester (GE Healthcare, Piscataway, N.J.) to chemically conjugate the Cy5.5 label on the sequences. The Cy5.5-labeled probes were further purified with a second HPLC purification. All probe identities were confirmed by electron spray ionization mass spectrometer (ESI-MS) using an Oligo HTCS system (Novatia LLC, Princeton, N.J.). The measured molecular weights are within 1.5 Daltons of the expected molecular weights. The purity of the probes was assessed with HPLC analysis and is typically greater than 90%. Quantitation of the probes was achieved by calculating from their UV absorption data and their nearest-neighbor-model-based extinction coefficients at 260 nm. Extinction coefficients of 2′-O-methyl-nucleotides and 2′-fluoro-nucleotides are assumed to be the same as that of RNA.

Breast Cancer Cell Lysates have High Nuclease Activity Against Probes

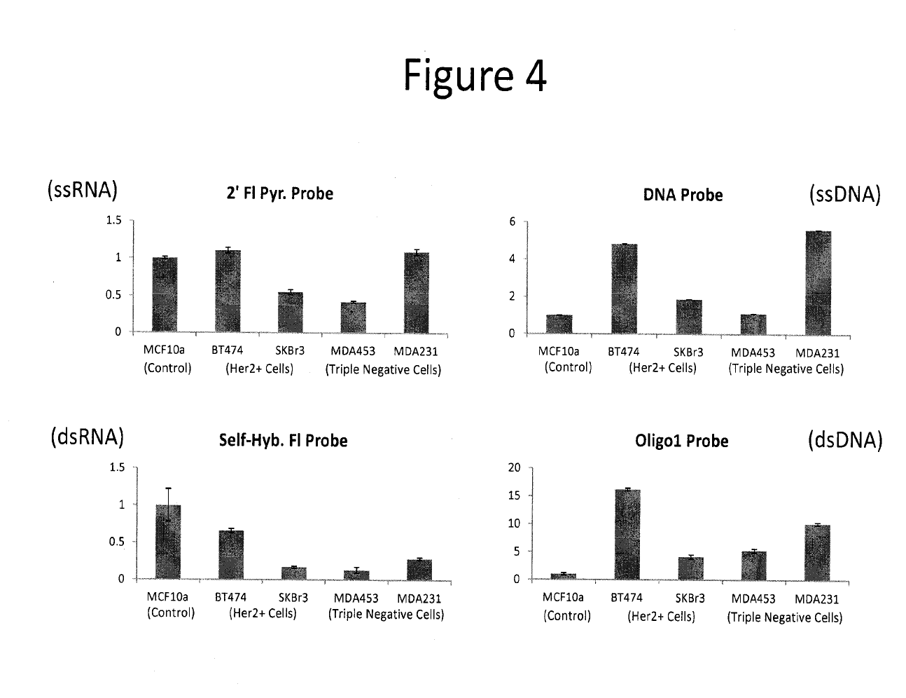

Cells from breast cancer cell lines or normal breast cell line were washed, lysed, and dialyzed against buffer containing 1 mM DTT, 1% Triton X-100, 50 mM Tris pH9, 150 mM NaCl, and 10 mM MgCl₂. Additionally, the buffer contains complete ULTRA protease inhibitor (Roche, product number 05892791001), at a concentration 1 tablet per 10 mL of buffer. The lysates were incubated with 50 pmol of probe, and fluorescence was measured after 1.5 hours. It was found that the breast cancer cell lysates did exhibit high nuclease activity against the probes. (FIG. 4).

Breast Cancer Cells Secrete Nucleases

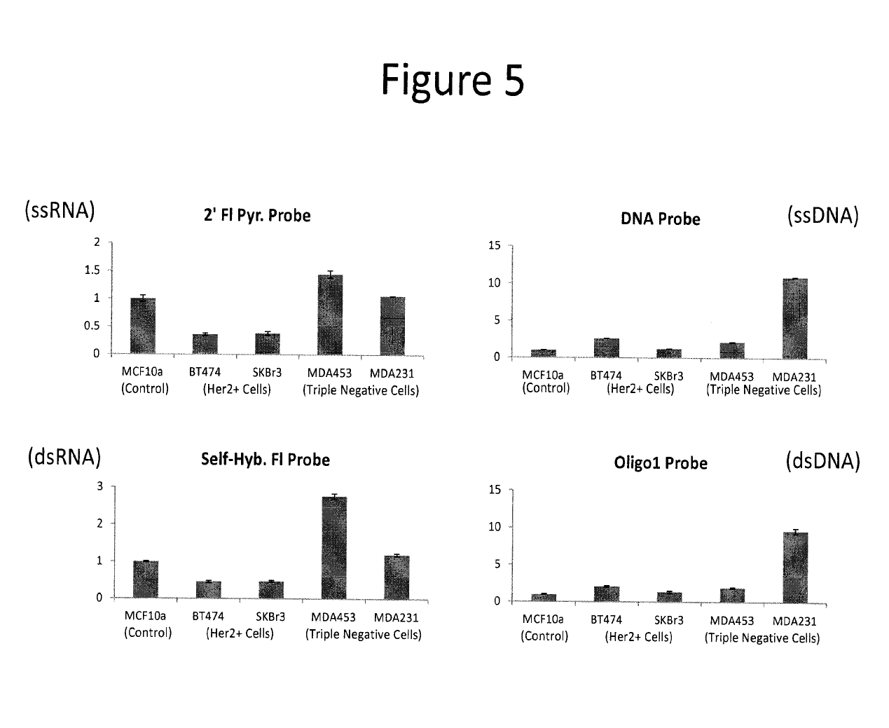

Next, it was examined whether breast cancer cells secrete nucleases. Breast cancer cell lines and normal breast cell lines ere incubated with serum free media overnight. The media was collected and the cell debris was spun down. The media was dialyzed against PBS+/+ with protease inhibitors, and the supernatants were incubated with 50 pmol of probe. Fluorescence was measured after 1.5 hours. It was found that the breast cancer cells did secrete nucleases. (FIG. 5).

Incubation Conditions

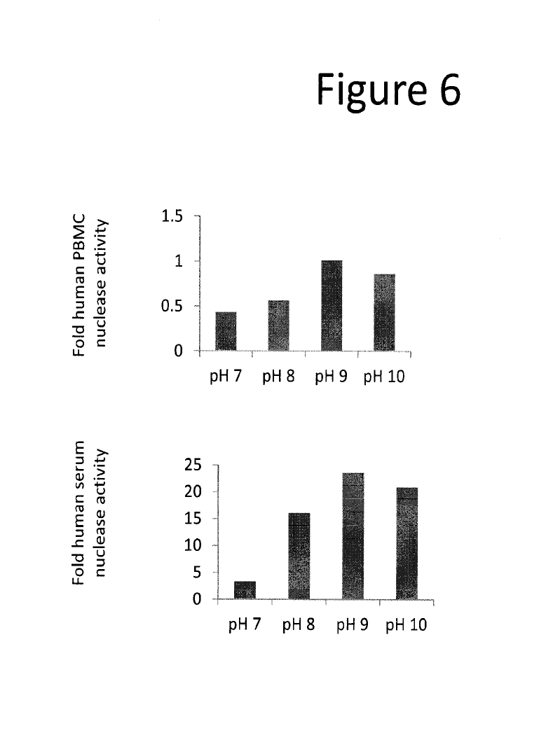

Incubation conditions were evaluated and optimized. MDA231, Human PBMC cell lysates, or human serum were dialyzed with Tris-buffer ranging from pH 7 to pH 10. A pH of 9 was found to be optimum (FIG. 6).

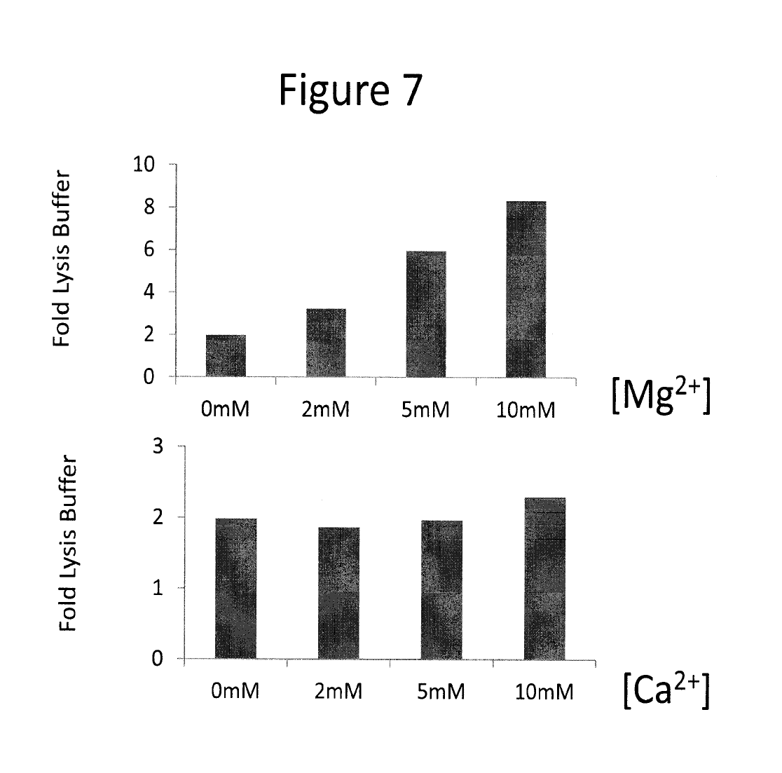

It was also evaluated whether the concentration of Ca²⁺ and/or Mg²⁺ affected the nuclease activity. MDA231 cell lysates were dialyzed with buffer containing various concentrations of Ca²⁺ or Mg²⁺, and nuclease activity against DNA probe was measured. It was found that the concentration of Ca²⁺ was not important, but that a concentration of 10 mM of Mg2⁺ was optimal (FIG. 7).

Controls

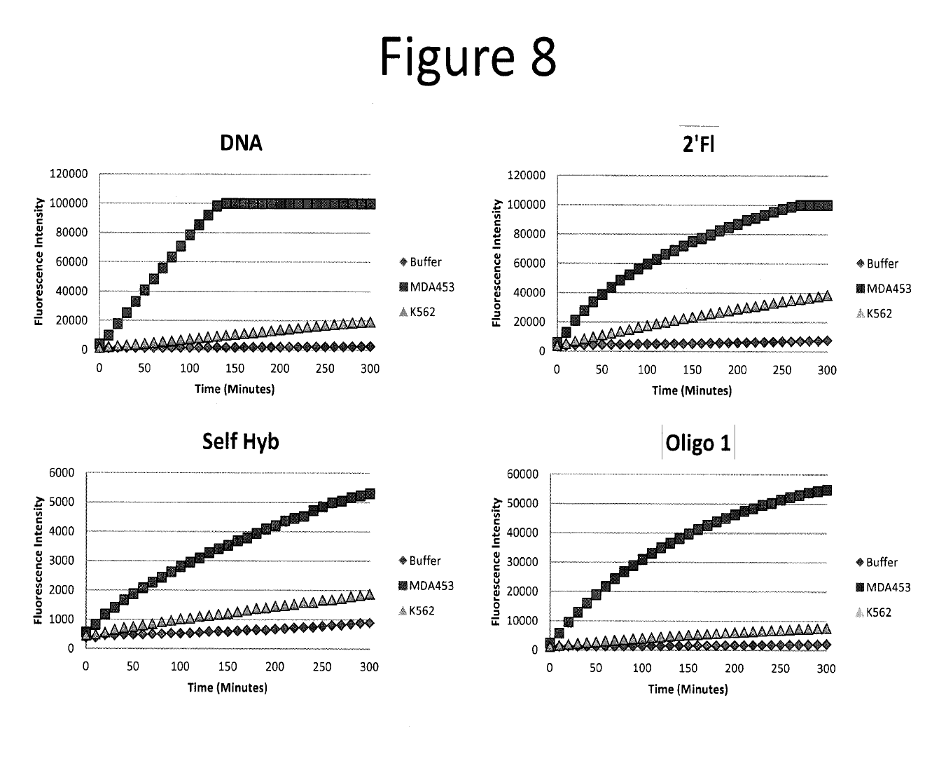

Both MCF10a (a mammary epithelia cell line) and K562 (hematopoietic cell line) cells were tested as possible controls. It was found that the K562 cells were better controls (FIG. 8).

Sensitivity

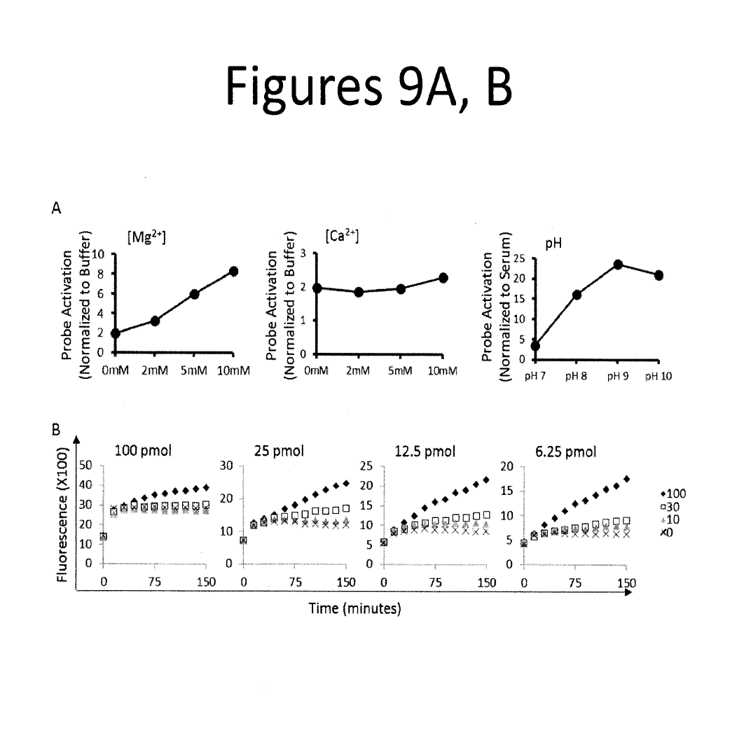

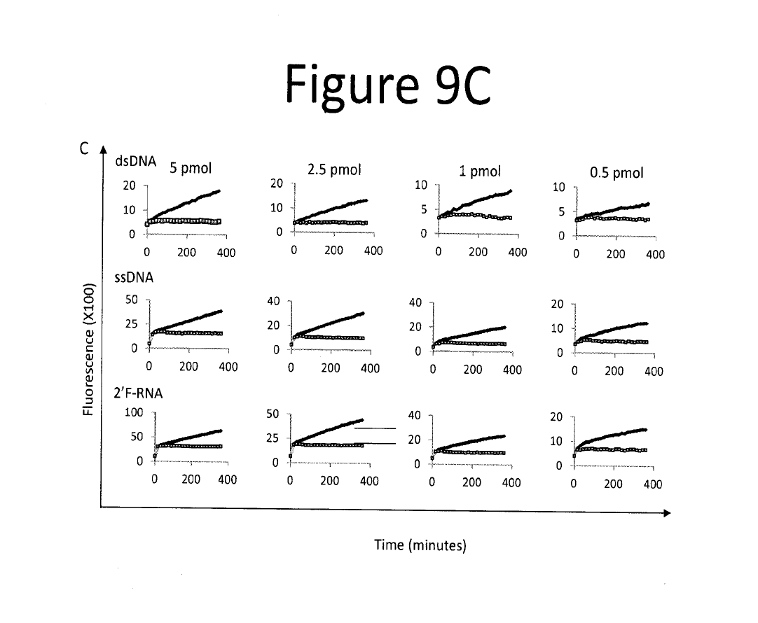

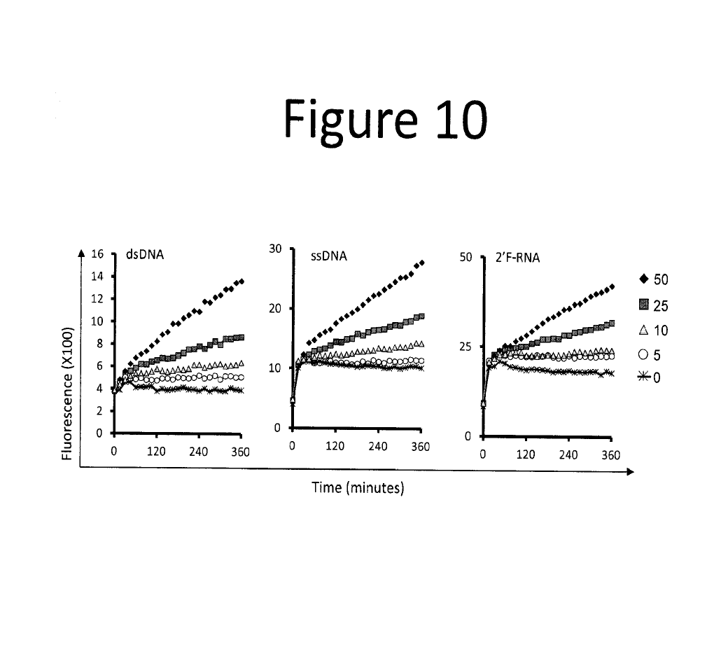

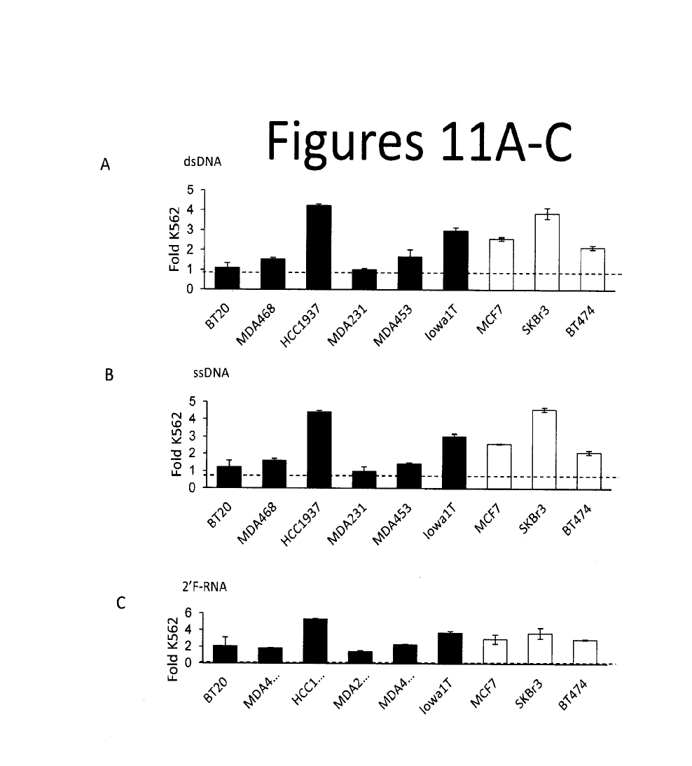

It was determined what is the lowest number of breast cancer cells detectable with these probes, and how much probe is optimal. Kinetics of nuclease degradation of different amounts of double stranded DNA (Oligo 1) probe (100 pmol, 25 pmol, 12.5 pmol, 6.25 pmol) by lysates of 100, 30, or 10 SKBr3 cells, or lysis buffer alone. Probes were digested 2.5 hours at 37 degrees C., with measurements taken every 15 minutes. It was determined that as few as 10 SKBr3 cells were detectable, and that using about 6.25 pmol of probe was effective (FIG. 9). It was determined that the present probes were effective for detecting several breast cancer subtypes (FIGS. 10 and 11).

Detection in Blood Samples

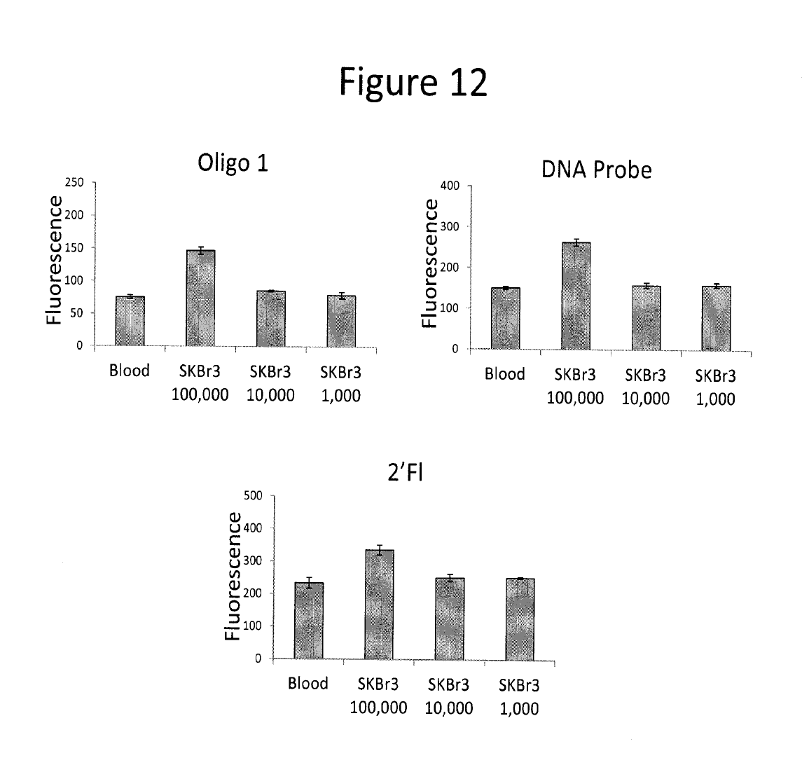

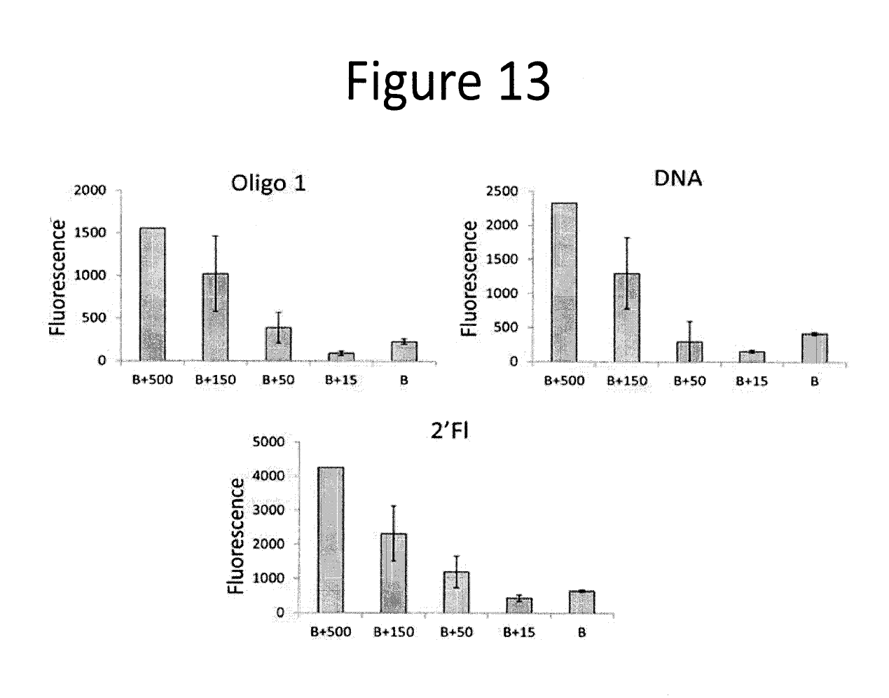

It was investigated whether breast cancer cell could be detected in blood using the present method. Briefly, 10⁵, 10⁴, or 10³ SKBr3 cells were spiked into 100 μL of human blood. The cells were pelleted cells and the plasma was removed. The cells were lysed with optimized buffer (described above) and tested for nuclease activity. It was possible to detect breast cancer cell could be detected in blood (FIGS. 12 and 13).

### Example 2

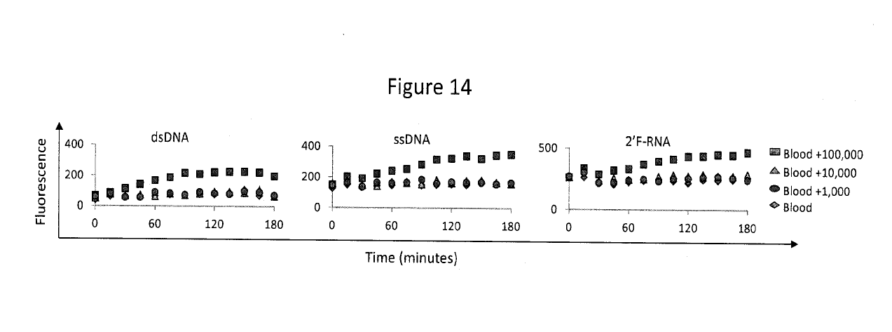

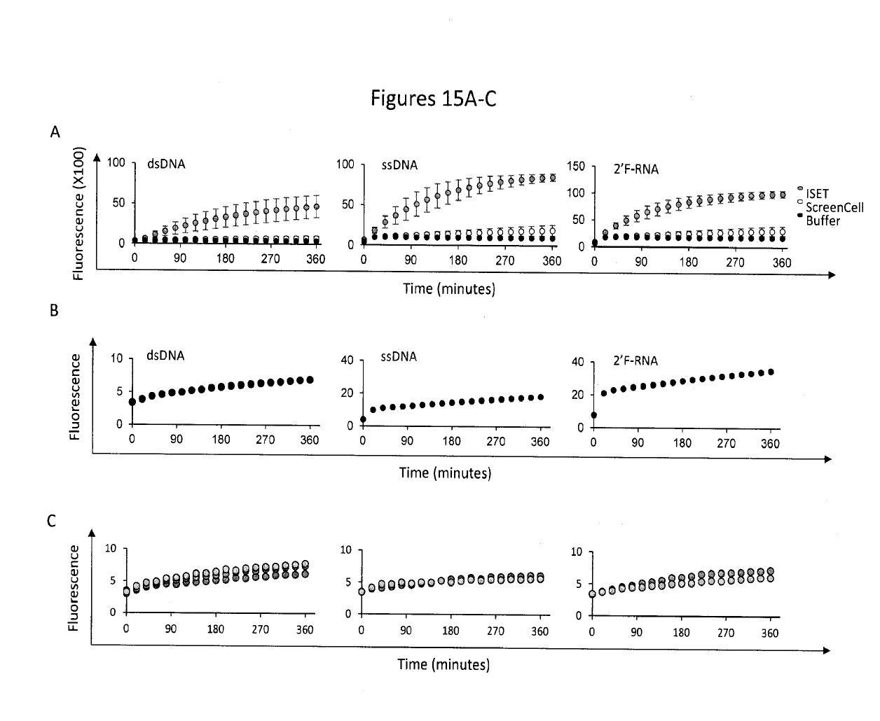

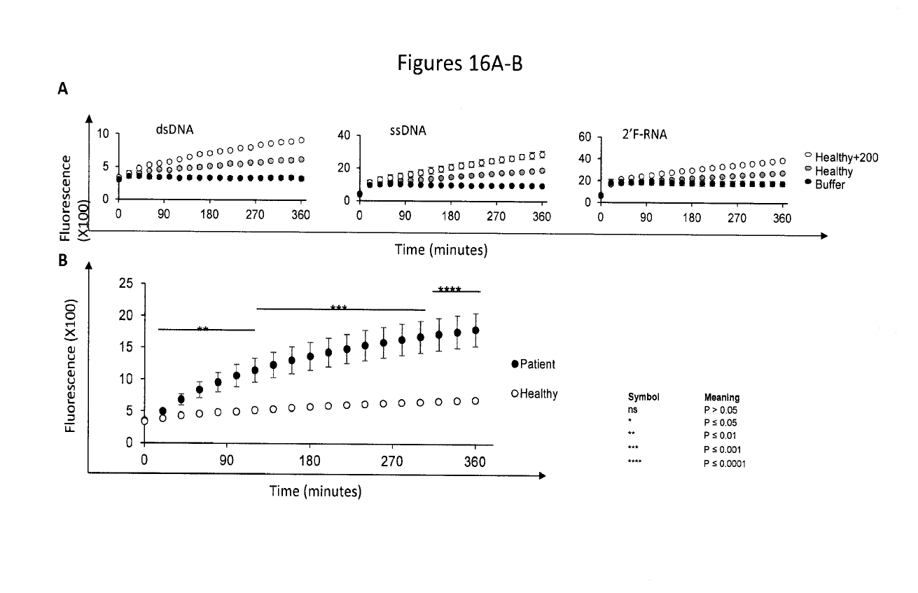

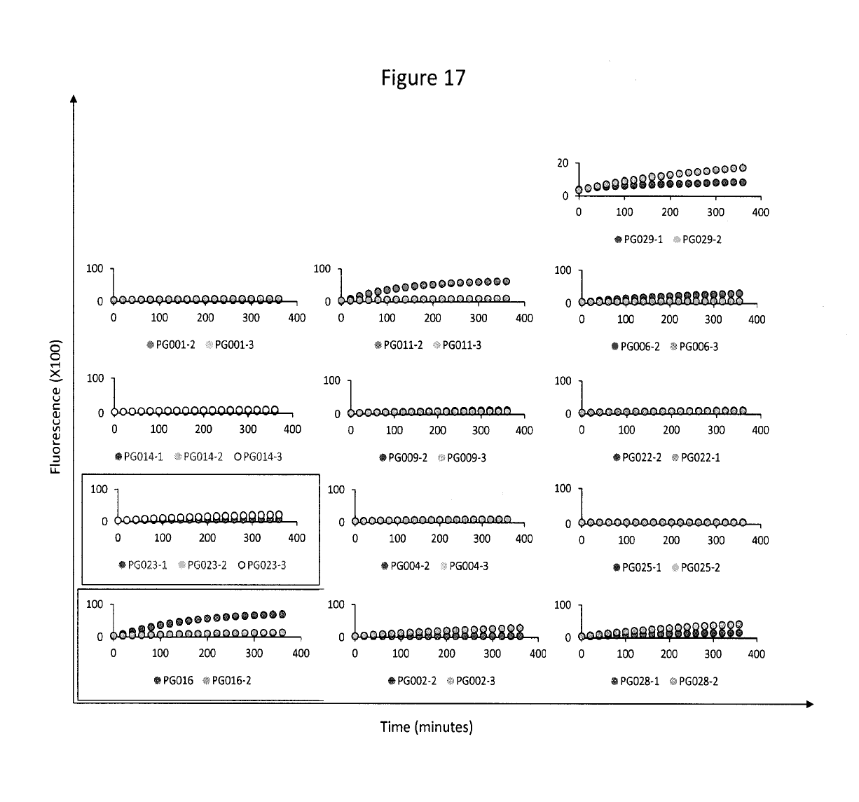

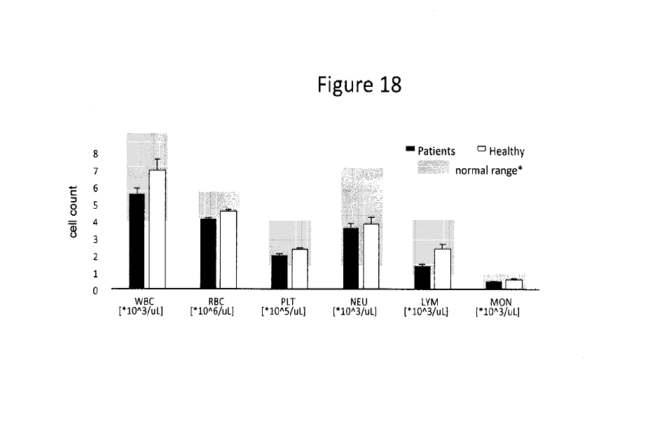

The sensitivity of the assay in blood is low due to background activity from blood cells (FIG. 14), which further confirmed the need for capturing/enriching CTCs from blood. CTCs can be enriched from blood using size exclusion filters. Two commercially available filters are the ISET filter from Rarecells and the filter from ScreenCell. The filter from ScreenCell was more effective at reducing the background signal from blood compared to the ISET filter (FIG. 15A). The variability in the background signal from blood derived from several healthy donors was examined (FIGS. 15 B and C). Fixed amounts of breast cancer cells were spiked in blood and process the mixture with the ScreenCell filters (FIG. 16A). It was possible to robustly detect 200 cancer cells spiked into 1 mL of blood, which was a 500 fold improvement in sensitivity over no filtration. The probes were validated in blood from patients with stage IV breast cancer. The probe used in this example was the dsDNA probe (FIG. 16B). The blood from patients and healthy donors was processed using the ScreenCell filter. These data clearly show a statistically significance between blood from patience with stage IV and healthy donor blood showing that the nuclease activated probes can successfully identify CTCs in patient samples. FIG. 17 shows the variability in probe activity from draw to draw. This variability could be due to the changes in potential response to treatment or disease progression. FIG. 18 shows that it was possible to rule out that the nuclease activity observed in the patient samples was due to higher amounts of blood cells present in the blood of these patients. Together, these data confirm that the nuclease activated robes can be used to detect CTC-nuclease activity in blood from patients with stage IV breast cancer.

### Example 3

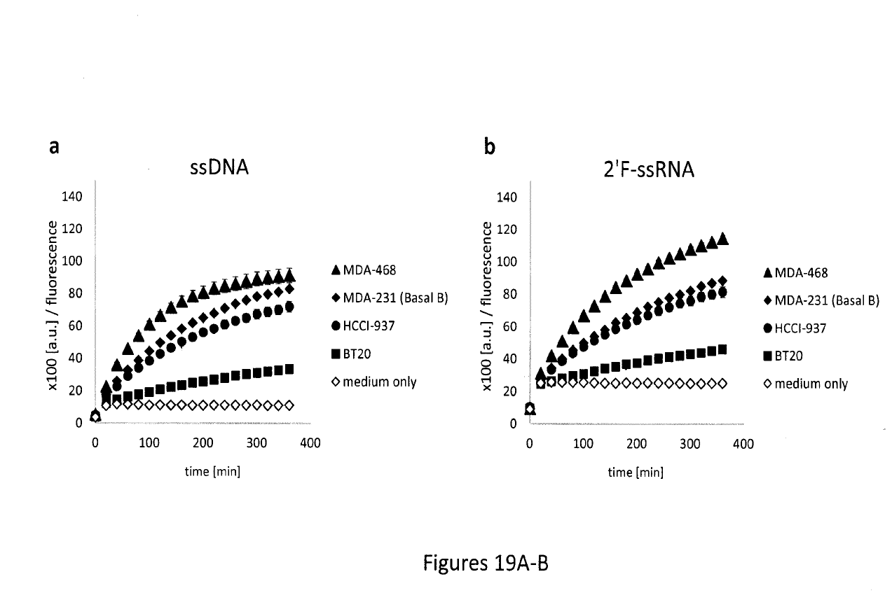

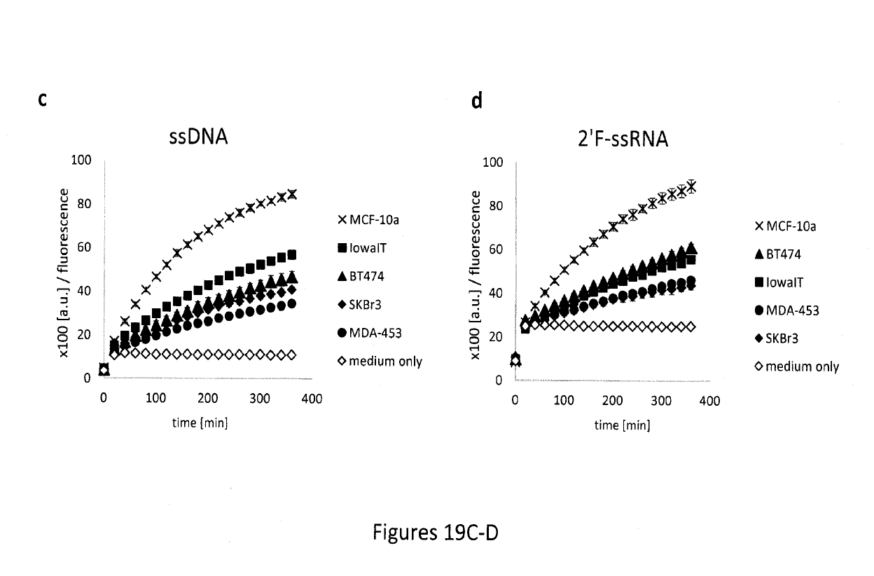

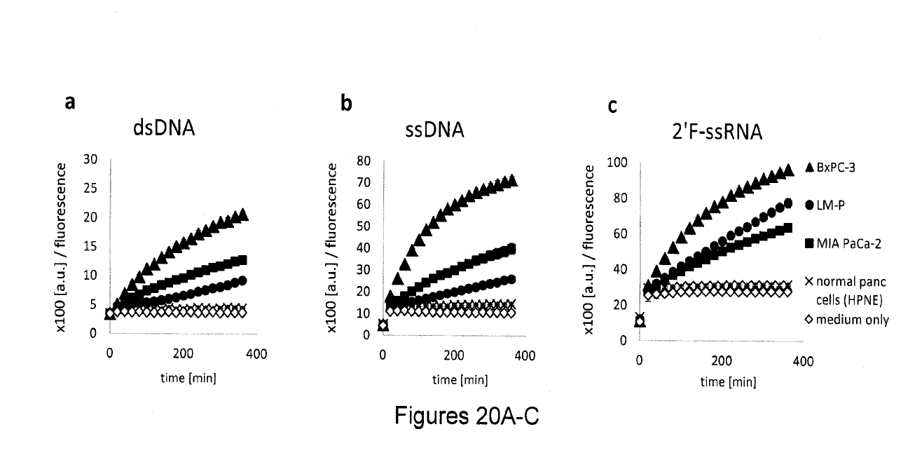

Experiments were performed to evaluate nuclease activity in supernatants from cancer cell lines. Secreted nuclease active was measured in contrast to intracellular nuclease activity. Experiments were performed to demonstrate that the ssDNA and 2′F-ssRNA nuclease activated probes were activated by secreted nucleases from breast cancer cells (FIGS. 19A-D and 20A-C). The data show that the probes can be used to detect nucleases that are secreted from CTCs in blood of breast cancer patients.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A substrate probe for detecting a circulating tumor cell (CTC) endonuclease comprising an oligonucleotide of 2-30 nucleotides in length, a fluorophore operably linked to the oligonucleotide, and a quencher operably linked to the oligonucleotide, wherein the oligonucleotide comprises one or more modified pyrimidines, is capable of being cleaved by a CTC nuclease but is resistant to cleavage by non-CTC nucleases.

2-3. (canceled)

4. The substrate probe of claim 1, wherein the oligonucleotide is 5′-CTACGTAG-3′ (SEQ ID NO:1), 5′-TCTCGTACGTAC-3′ (SEQ ID NO:2), 5′-CUACGUAG-3′ (SEQ ID NO:3) or 5′-UCUCGUACGUAC-3′ (SEQ ID NO:4).

5. (canceled)

6. The substrate probe of claim 1, wherein one or more of the pyrimidines are chemically modified.

7. The substrate probe of claim 6, wherein one or more of the pyrimidines are 2′-O-methyl modified and/or are 2′-fluoro modified.

8. (canceled)

9. The substrate probe of claim 1, wherein one or more of the purines, if present, are chemically modified.

10. The substrate probe of claim 9, wherein one or more of the purines are 2′-O-methyl modified and/or are 2′-fluoro modified.

11. (canceled)

12. The substrate probe of claim 1, wherein the fluorophore is selected from the group consisting of the fluorophores listed in Table 1.

13. (canceled)

14. The substrate probe of claim 1, wherein the quencher is selected from the group consisting of the quenchers listed in Table 2.

15. The substrate probe of claim 1, wherein the oligonucleotide is single-stranded.

16. The substrate probe of claim 1, wherein the oligonucleotide comprises both RNA and DNA.

17. A method of detecting a circulating tumor cell (CTC) in a sample comprising measuring fluorescence of a sample that has been contacted with a substrate probe of claim 1, wherein a fluorescence level that is greater than the fluorescence level of a control indicates that the sample has a CTC.

18-19. (canceled)

20. The method of claim 17, wherein the CTC is a metastatic breast cancer cell.

21. The method of claim 17, wherein the fluorophore is FAM, Cy5, Cy5.5, Cy7, Licor IRDye 700, Cy7.5, Dy780, Dy781, DyLight 800, Licor IRDye 800 CW, or Alexa Fluor 647, 660, 680, 750, or 790.

22. (canceled)

23. The method of claim 17, wherein the fluorophore is FAM, TET, HEX, JOE, MAX, Cy3, or TAMRA and the quencher is IBFQ, BHQ1, BHQ2, or Licor IRDye QC-1.

24. The method of claim 17, wherein the fluorophore is ROX, Texas Red, Cy5, or Cy5.5 and the quencher is IBRQ or BHQ2.

25. A method for detecting CTC nuclease activity in a test sample, comprising:
(a) Contacting the test sample with a composition comprising substrate probe of claim 1, thereby creating a test reaction mixture,
(b) incubating the test reaction mixture for a time sufficient for cleavage of the substrate probe by a CTC nuclease in the sample; and
(c) Determining whether a detectable fluorescence signal is emitted from the test reaction mixture, wherein emission of a fluorescence signal from the reaction mixture indicates that the sample contains CTC nuclease activity.

26. The method of claim 25, wherein the composition further comprises a buffer at pH8 to pH10.

27. (canceled)

28. The method of claim 25, wherein the composition further comprises Mg²⁺ at a concentration of 2 mM to 20 mM.

29. (canceled)

30. The method of claim 25, wherein the composition lacks Triton X-100.

31. A method for detecting CTC nuclease activity in a test sample, comprising:
(a) Contacting the test sample with a composition comprising substrate probe of claim 1, thereby creating a test reaction mixture,
(b) Incubating the test reaction mixture for a time sufficient for cleavage of the substrate probe by a CTC nuclease in the sample; and
(c) Determining whether a detectable fluorescence signal is emitted from the test reaction mixture;
(d) Contacting a control sample with the substrate probe, wherein the control sample comprises a predetermined amount of nuclease, thereby creating a control reaction mixture;
(e) Incubating the control reaction mixture for a time sufficient for cleavage of the substrate probe by a nuclease in the control sample; and
(f) determining whether a detectable fluorescence signal is emitted from the control reaction mixture; wherein detection of a greater fluorescence signal in the test reaction mixture than in the control reaction mixture indicates that the test sample contains greater nuclease activity than in the control sample, and wherein detection of a lesser fluorescence signal in the test reaction mixture than in the control reaction mixture indicates that the test sample contains less nuclease activity than in the control sample.

32. (canceled)

33. The method of claim 31, further comprising contacting the test sample with a buffer before or during step (a).

34-36. (canceled)


SEQUENCE LISTING (4 sequences)

SEQ ID NO: 1 - DNA, 8 bp, Artificial Sequence
  Description of Artificial Sequence Synthetic probe
ctacgtag

SEQ ID NO: 2 - DNA, 12 bp, Artificial Sequence
  Description of Artificial Sequence Synthetic probe
tctcgtacgtac

SEQ ID NO: 3 - RNA, 8 nt, Artificial Sequence
  Description of Artificial Sequence Synthetic probe
cuacguag

SEQ ID NO: 4 - RNA, 12 nt, Artificial Sequence
  Description of Artificial Sequence Synthetic probe
ucucguacguac